(19) United States
(12) Patent Application Publication
(10) Pub. No.: US 2014/0024811 A1
(43) Pub. Date: Jan. 23, 2014

(54) CANCER DETECTION

(71) Applicant: John R. Jenkins, Liverpool (GB)

(72) Inventor: John R. Jenkins, Liverpool (GB)

(73) Assignee: THE UNIVERSITY OF LIVERPOOL, Liverpool (GB)

(21) Appl. No.: 14/008,241

(22) PCT Filed: Mar. 29, 2012
(86) PCT No.: PCT/GB12/50711

§ 371 Date: Sep. 27, 2013

(30) Foreign Application Priority Data
Mar. 29, 2011 (GB) 1105295.8

(51) Int. Cl. G01N 33/574; C07K 16/18; C12Q 1/68

(52) U.S. Cl. CPC G01N 33/574; C12Q 1/68; C12Q 1/686; C07K 16/18
USPC 530/389.7; 435/7.92; 435/7.94; 435/7.23; 435/6.14; 435/6.12; 506/7; 436/501

(57) ABSTRACT

Provided are methods of detecting cancer or pre-cancerous conditions in patients comprising assaying a patient sample for an elevated level of target molecules representative of expression of nucleosome assembly protein 1-like 1 (NAP1L1), wherein elevated levels of expression of NAP1L1 are indicative of a cancer of the colon, or a precancerous condition of the colon. Also provided are similar methods using panels of biomarkers such as HMGB1; PHB; RPL6; NAP1L1 and CK18. The invention also provides a method for assessing effectiveness of a therapy or putative therapy, methods of staging cancer and assessing progression of a cancer and methods of determining an appropriate cancer treatment regimen.

The present invention relates to methods of detecting cancer or pre-cancerous conditions in patients. The invention also relates to methods of staging cancer and assessing progression of a cancer. In other embodiments the invention relates to methods of determining an appropriate cancer treatment regimen.

Cancer accounts for more than a tenth of all mortality worldwide, and was responsible for 7,600,000 deaths in 2008. Of this total, stomach cancer, colorectal cancer and breast cancer account for nearly 2,000,000 deaths.

Colon cancer is one of the leading causes of cancer mortality, and incidences of colon cancer peak in patients between the ages of 60 and 70. Survival rates are markedly improved in patients where cancer is detected at an early stage of progression. Unfortunately, the majority of cases are detected at a relatively late stage and this has adverse consequences on rates of patient survival.

The standard screening programme for detection of bowel cancer (in the UK at least) makes use of a faecal occult blood (FOB) test. This is used as a preliminary screen to identify patients where further investigation is considered advisable. Current statistics indicate that of every 1,000 FOB tests completed approximately 20 will be positive, and of this 20 approximately 16 patients will undergo subsequent colonoscopy with a view to identifying the cause of bleeding. Of patients undergoing colonoscopy, approximately 40% will have a colonic adenoma (a pre-cancerous condition that is a precursor to colon cancer), while approximately 10% will have a colonic adenocarcinoma. This programme of FOB testing followed by colonoscopy has been shown to reduce mortality rates by approximately 15%, since it allows earlier identification, and so earlier treatment, of colorectal cancer.

Unfortunately, there are significant drawbacks to current screening and investigative mechanisms. The FOB tests used for preliminary screening may generate both false negative and false positive results. Furthermore, colonoscopy is an invasive procedure that can be uncomfortable and embarrassing, and also has a relatively high incidence of more serious complication. Approximately 1 in 1,000 colonoscopy patients experience complications, while 1 in 10,000 die as a result of the procedure.

Not only are the current screening and investigate techniques technically far from ideal, but they are also expensive. The bowel cancer screening programme undertaken between 2008 and 2009 in the UK cost an estimated £55 m, and this cost is likely to increase as the age range of participants is broadened, and as the population grows older.

In light of the above it can be seen that there is a need for improved methods by which cancerous and pre-cancerous conditions can be detected. There is also a need for new and/or improved methods by which early stage cancers or pre-cancerous conditions can be detected. Furthermore, there is a need for new and/or improved methods of staging cancers. Methods capable of staging the early progression of cancer are likely to be of particular value since they will allow early therapeutic intervention. There is also a need for new and/or improved methods of determining prognosis for survival of cancer patients. Furthermore, there is a need for methods of selecting regimens for the treatment of cancer patients, and also for the provision of new medicines that may be used in the treatment and/or prevention of cancers.

It is an aim of certain aspects and embodiments to alleviate at least some of the failings associated with the prior art.

In a first aspect the invention provides a method of detecting a cancer of the colon, or a precancerous condition of the colon, the method comprising assaying a patient sample for an elevated level of target molecules representative of expression of nucleosome assembly protein 1-like 1 (NAP1L1), wherein an elevated level of the target molecules representative of the expression of NAP1L1 is indicative of a cancer of the colon, or a precancerous condition of the colon.

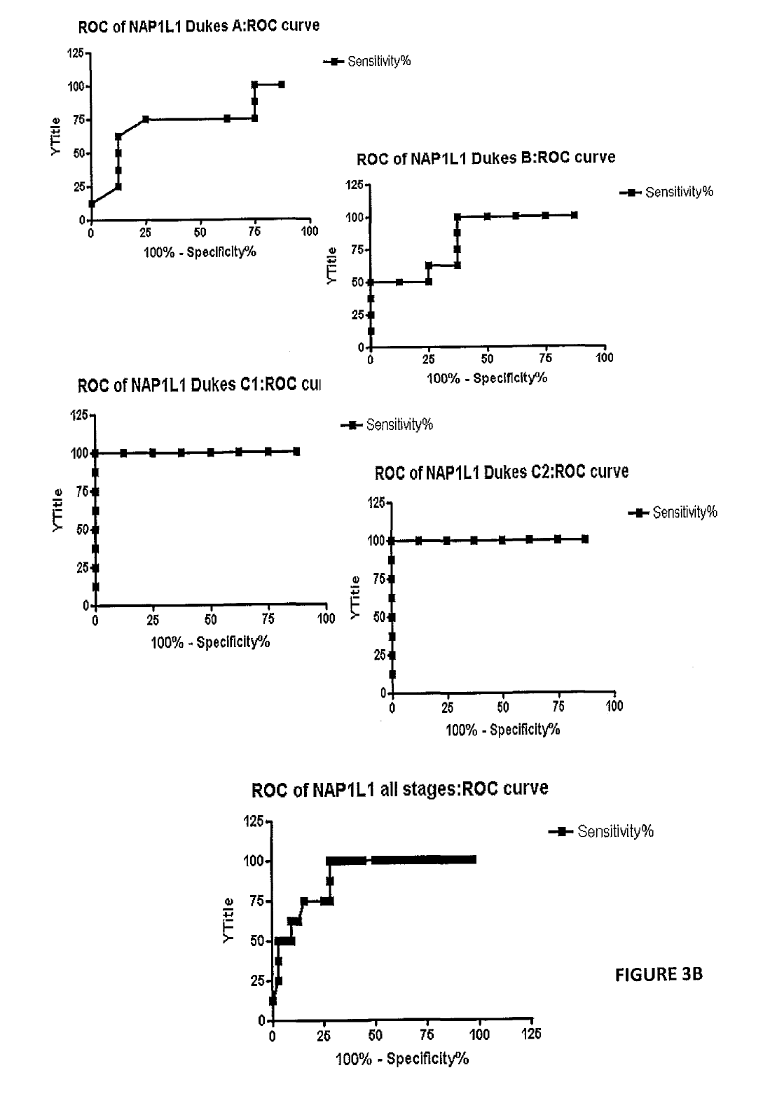

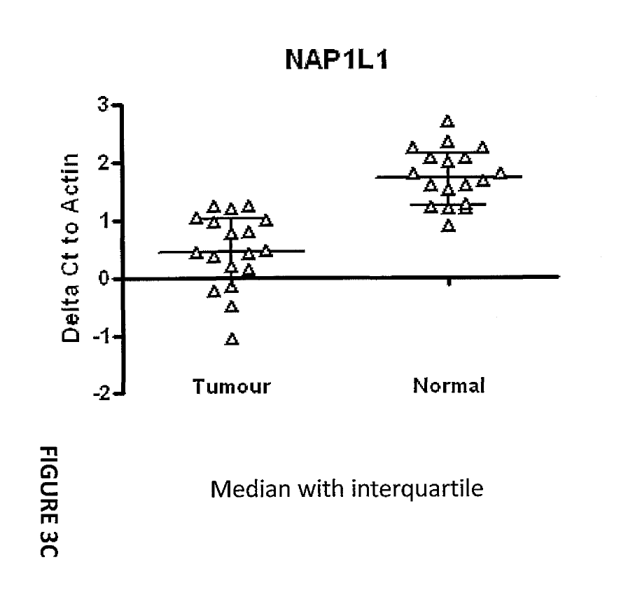

Increased expression of NAP1L1 in a patient sample has been found to provide a surprising strong indication of the presence of a cancer of the colon, or a precancerous condition of the colon. As discussed elsewhere in the specification, and shown in FIG. 3C, statistical analysis indicates that increased expression of NAP1L1 in serum samples serves as a biomarker for colorectal cancer that exhibits a ROC (Receiver Operating Characteristic) of 0.8906 and a P value of 0.0007271. The skilled person will readily recognise that these values clearly illustrate the remarkable utility of this biomarker in the detection of cancer. This ability is even more remarkable since prior art studies have suggested that NAP1L1 lacks a diagnostic potential in colorectal cancer patients (Chan, et al., “Multiple serological biomarkers for colorectal cancer detection”), and that NAP1L1 expression is not significantly increased in colorectal carcinomas (in direct contrast to the inventor's findings underlying the present invention), instead only being increased in small intestine carcinoids (Kidd, et al. “The Role of Genetic Markers—NAP1L1, MAGE-D2 and MTA1—in Defining Small-Intestinal Carcinoid Neoplasia”).

The skilled person will also realise that the ability to detect statistically significant changes in NAP1L1 expression by assessment samples from patients such as serum, tissue, or blood, offers marked advantages. One advantage associated with the use of blood or serum samples, is that these can be obtained without the need for invasive surgery or colonoscopy. The data presented here further provide a clear indication that other sample types, particularly those containing or directly derived from colonic or colorectal tissue, share the same utility.

Although the methods of this first aspect of the invention may be practiced using NAP1L1 as the sole biomarker for cancer of the colon (or a precancerous condition of the colon), these methods may also make us of further biomarkers. In such an embodiment, a method of the first aspect of the invention may further comprise assaying the patient sample for an elevated level of a further target molecule representative of expression of a gene selected from the group consisting of: RPL6; and PHB, wherein elevated levels of the target molecules representative of the expression of NAP1L1 and the further target molecules are indicative of a cancer of the colon, or a precancerous condition of the colon. Indeed, in certain embodiments, such methods may comprise assaying the patient sample for both further target molecules representative of expression of RPL6 and further target molecules representative of expression of PHB.

In a second aspect the invention provides a method of detecting a cancer or a pre-cancerous condition, the method comprising:

assaying a patient sample for an elevated level of target molecules representative of expression of at least two genes encoding proteins selected from the group consisting of: HMGB1; PHB; RPL6; NAP1L1 and CK18;
wherein the elevated level of the target molecules is indicative of a cancer or pre-cancerous condition in the patient.

The cancer to be detected by a method in accordance with this second embodiment of the invention may be an early stage cancer. Alternatively or additionally, the cancer or precancerous condition may be a cancer of the colon, or a precancerous condition of the colon.

For the purposes of the present invention, a “cancer of the colon” should be taken as encompassing at least colon cancer and/or colorectal cancer. A “precancerous condition” should be taken as encompassing any condition that is clinically viewed as representing a precursor to cancer, such as adenoma, adenocarcinoma, or the like.

Embodiments of the methods of the first aspect of the invention involving assaying for expression of RPL6 and/or PHB, or methods in accordance with the second aspect of the invention, may offer greater reliability, Those methods of the invention utilising PHB as a biomarker may be advantageous in circumstances in which it is wished to determine a suitable mode of treatment for patients in whom cancer, or a precancerous condition, has been detected. These include better performance characteristics and increases in sensitivity and in specificity of such methods. These benefits are applicable both to methods of the invention used for the detection of cancer (or precancerous conditions) and to methods of the invention used for staging of cancer.

In a third aspect, the invention provides a method for assessing effectiveness of a therapy or putative therapy, the method comprising:

determining a level of expression of NAP1L1 in a sample representative of gene or protein expression in a sample of a cancer;
providing the therapy or putative therapy to a sample of a cancer;
determining a level of expression of NAP1L1 in a sample representative of gene expression in a sample of the cancer to which the therapy or putative therapy has been provided.

In certain embodiments the method may comprise further determining levels of expression of one or more further genes or proteins selected from the groups described elsewhere in the specification. In certain embodiments such further genes may comprise PHB and/or RPL6.

In certain embodiments the cancer sample is an in vitro cancer sample. In certain embodiments the cancer sample is from a cancer patient undergoing treatment with the therapy or putative therapy.

In certain embodiments the therapy or putative therapy may comprise a therapeutic agent or therapeutic treatment regimen.

In a further aspect the invention provides a method of detecting cancer or a pre-cancerous condition, the method comprising:

assaying a patient sample for an elevated level of target molecules representative of expression of at least two genes encoding proteins selected from the group consisting of:

### HMGB1; PHB; and CK18;

wherein an elevated level of the target molecules in the patient sample is indicative of a cancer or pre-cancerous condition in the patient.

Methods in accordance with this aspect of the invention may, in certain embodiments, also include assaying a patient sample for an elevated level of a target molecule representative of expression of the gene encoding FABP6.

Suitably the elevated level of the target molecule representative of gene expression may be assessed by comparing the amount of the target molecule present in the patient sample under investigation with a reference value indicative of the amount of the target molecule in a control sample. Suitable control samples may be derived from individuals without cancer, and preferably may be age matched with the patient undergoing investigation. Suitable control samples may also be derived from matched non-cancerous tissues. It will be appreciated that it is not necessary for analysis of target molecules in a control sample to be undertaken each time that a method in accordance with this aspect of the invention is performed, since a suitable reference value may be determined from the results of previous analyses.

In a further aspect of the invention, there is provided a method of detecting cancer or a pre-cancerous condition, the method comprising:

assaying a patient sample for an elevated level of target molecules representative of expression of the group of genes consisting of:

### HMGB1, RPL6, NCL, PHB, NPM, CK18, NAP1L1, SFRS2, FABP6, DDX5, CBX3

wherein the presence of the target molecules representative of expression of these genes is indicative of a cancer or pre-cancerous condition in the patient.

The inventors have surprisingly found that cancers expressing this extensive panel of markers tend not to be particularly aggressive. This information may be useful in determining how best to treat cancers of these patients.

In a further aspect the invention provides a method of staging a cancer, the method comprising:

assaying a sample from a patient with cancer for the presence of target molecules representative of expression of at least two genes encoding protein selected from the group consisting of:

### HMGB1, RPL6, NCL, PHB, CK18, NAP1L1, FABP6, DDX5, CBX3

wherein an elevated level of the target molecule representative of expression of the genes indicates the stage of the patient's cancer.

In a further aspect the invention provides a method of staging a cancer, the method comprising:

assaying a sample from a patient with cancer for the presence of target molecules representative of expression of a gene encoding a protein selected from the group consisting of:

### HMGB1, RPL6, NCL, PHB, CK18, NAP1L1, FABP6, DDX5, CBX3

and using the information regarding the expression of these genes to stage the patient's cancer.

A method of staging a cancer in accordance with the preceding aspect of the invention may optionally include assaying the patient sample for target molecules representative of expression of NPM and/or SFRS2.

An increase in expression across the sum of the genes assessed may be indicative that the patient's cancer is at a stage equivalent to stage B of the Dukes' classification system for colorectal cancer.

Elevated expression of a subgroup of genes selected from a subgroup comprising HMGB, NCL, PHB and CK18 may indicate that the patient's cancer is at a stage equivalent to Dukes C.

Elevated expression of genes selected from a subgroup comprising NAP1L1; RPL6 and HMGB1 may be indicative that the patient's cancer is at a stage equivalent to stage A of the Dukes' classification system. It will be appreciated that the ability to identify cancers at such an early stage of progression is of benefit since it allows early therapeutic intervention, which markedly increases the chance of successfully treating the cancer (whether by surgical intervention or other therapeutic means). Cancers at an early stage of progression equivalent to Dukes' stage A or B can be removed locally, without the need for major surgery or large scale excision of the bowel.

So useful is the ability to use sub-groups of the biomarkers identified to allow early staging of cancer that it gives rise a further aspect of the invention—a method of staging a cancer, the method comprising:

assaying a sample from a patient with cancer for the presence of target molecules representative of expression of a gene encoding protein selected from the group consisting of:

### HMGB1, RPL6, and NAP1L1,

and using the information regarding the expression of these genes to stage the patient's cancer.

In a further aspect, the invention provides a method of determining a prognosis for survival of a cancer patient, the method comprising:

assaying a sample from a patient with cancer for the presence of target molecules representative of expression of SFRS2 and/or NPM;
wherein an elevated level of the target molecules in the sample is indicative of a good prognosis.

In a further aspect, the invention provides a method of determining a prognosis for survival of a cancer patient, the method comprising:

assaying a sample from a patient with cancer for the presence of target molecules representative of expression of PHB and/or CK18;
wherein an elevated level of the target molecules in the sample is indicative of a good prognosis.

An further aspect of the invention provides a method of determining a prognosis for survival of a cancer patient, the method comprising:

assaying a sample from a patient with cancer for the presence of target molecules representative of expression of:
SFRS2 and/or NPM; and
HMGB1 and/or PHB and/or CK18
   wherein a decreased level of the target molecules representative of expression of SFRS2 and/or NPM, and an elevated level of the target molecules representative of expression of PHB and/or CK18 is indicative of a poor prognosis.

A further aspect of the invention provides a method of selecting a cancer treatment regimen, the method comprising:

assaying a sample from a patient with cancer for the presence of target molecules representative of expression of:
SFRS2 and/or NPM; and
HMGB1 and/or PHB and/or CK18
   wherein a decreased level of the target molecules representative of expression of SFRS2 and/or NPM, and an elevated level of the target molecules representative of expression of PHB and/or CK18 indicates that the patient would benefit from an aggressive cancer treatment regimen.

This preceding aspect of the invention is based upon the inventors' surprising finding that patients expressing one or more of the markers HMGB1, PHB or CK18, and who do not also express one or both of SFRS2 or NPM, tend to have very short survival times. Thus, even if the cancer is at an early stage of progression it may be beneficial to treat the cancer in an aggressive manner since failure to do so may notably impair patient survival. An aggressive treatment regime in accordance with this aspect of the invention may, for example, include surgery to remove much or all of the tissue in which cancer is present. Thus, in the case that the cancer is colorectal cancer an aggressive cancer treatment regimen may involve removal of the colon. The selection of cancer treatment regimes in accordance with this aspect of the invention provides an individual determining the treatment of a cancer patient with information as to whether or not the patient in question has a form of cancer that is associated with low rates of patient survival. This information allows a decision regarding aggressive treatment to be taken at an earlier point in cancer progression than would otherwise be the case.

In a further aspect the invention provides an inhibitor of HMGB1 activity for use as a medicine for the prevention of cancer of the digestive tract. Inhibitors of HMGB1 activity for use in accordance with this aspect of the invention may be used prophylactically, for example in patients with a pre-cancerous condition, to prevent the development of cancer. Inhibitors for use in accordance with this aspect of the invention may thus be used as medicines for provision to patients at risk of the development of cancer (such as colorectal or stomach cancer), such as patients with a pre-cancerous condition. Alternatively, inhibitors for use in accordance with this aspect of the invention may be used in medicaments for administration to patients whose cancer is at a very early stage of development, for example shortly after the development of cancer from a pre-cancerous condition, where the medicament may serve to prevent further development of the cancer. The inhibitor of HMGB1 activity may suitably be an inhibitor of the cytokine activity of HMGB1. An inhibitor suitable for use in accordance with this aspect of the invention may be able to inhibit HMGB1 activity extracellularly. Merely by way of example, suitable inhibitors may include neutralising antibodies.

Methods in accordance with the present invention may be of use in the detection, staging, prognosis and selection of regimens for treatment of Wnt-driven cancers. Merely by way of example, the inventors believe that methods in accordance with the invention may be used in the detection, staging, prognosis and selection of regimens for treatment of Wnt-driven cancers (or where appropriate pre-cancerous conditions) of the digestive tract, such as Wnt-driven gastrointestinal cancers including stomach cancer colorectal cancer. Methods in accordance with the invention may also be used in the detection, staging, prognosis and selection of regimens for treatment of Wnt-driven cancers (or where appropriate pre-cancerous conditions) such as breast cancer, lung cancer, liver cancer, ovarian cancer, neurological cancers, and skin cancer (for example melanoma).

The methods of the invention may be particularly useful in detection, staging, and selection of treatment regimens for cancers of the digestive tract, in particular cancers of the gastrointestinal tract such as colorectal cancer or stomach cancer.

For the purposes of the present disclosure the following protein nomenclature has been used:

HMGB1 is used to refer to “high-mobility group box 1”.

RPL6 is used to refer to “ribosomal protein L6”.

NCL is used to refer to “nucleolin”.

PHB is used to refer to “prohibitin”.

NPM is used to refer to “nucleophosmin”.

CK18 is used to refer to “cytokeratin 18”.

NAP1L1 is used to refer to “nucleosome assembly protein 1-like 1”.

SFRS2 is used to refer to “serine/arginine-rich splicing factor 2”.

FABP6 is used to refer to “fatty acid binding protein 6”.

DDX5 is used to refer to “DEAD box polypeptide 5”.

CBX3 is used to refer to “chromobox homologue 3”.

Further details of these proteins, including alternative names and external identifiers are provided in the Appendix.

The present invention is based upon the inventors' surprising finding that these 11 proteins represent a panel of biomarkers with particular utility in the detection of cancer and pre-cancerous conditions, cancer staging, prognosis, or selection of treatment regimens. While the panel as a whole has utility in the detection of cancer, there are also certain sub-groups within this panel that have further surprising properties that give rise to various of the other aspects of the invention described therein.

While individual biomarkers associated with colorectal cancer have been disclosed in the prior art, there has been no previous indication regarding the particular utility of the panel of biomarkers identified for the first time in the present specification, nor of the uses of the various sub-groups of this panel.

As described in the various aspects of the invention, the inventors have identified that sub-groups within this panel have particular utilities. For example, assaying for markers selected from the group consisting of HMGB1, PHB, and CK18 represents a preferred selection from within the broader panel in the event that it is wished to detect the presence of cancer in a patient. Markers selected from the group consisting of HMGB1, RPL6, NAP1L1 (used either in addition, or as an alternative, to the markers identified in the preceding sentence) represent a preferred selection from within the broader panel in the event that it is wished to detect the presence of a pre-cancerous condition (or very early stage of cancer) in a patient. These selected sub-groups (and particular embodiments in which combinations within these sub-groups are investigated) are distinct from the prior art, and provides advantages that are not known from the prior art.

Similarly, it has never previously been recognised that expression of one or more markers selected from the group consisting of SFRS2 and NPM provide a particularly strong indication that a cancer patient will have good prospects of survival, or that markers selected from the group consisting of HMGB1, PHB, and CK18 are all indicative of particularly dangerous forms of cancer with reduced patient survival times.

In certain embodiments of the methods of the invention the method may involve assaying the patient sample for the presence of target molecules representative of the expression of more than one of the biomarkers identified by the inventors. For example, the methods of the invention may involve assaying the sample for the presence of target molecules representative of expression of genes encoding all three of the proteins in the group comprising HMGB1; PHB and CK18. In certain embodiments the methods of the invention may involve assaying the sample for the presence of target molecules representative of expression of genes encoding each of the proteins HMGB1; PHB and CK18, and optionally one or more other genes from the broad panel identified.

In certain embodiments, the methods of the invention may further comprise assaying a sample from the same patient (which may be, but need not necessarily be, the same sample) an elevated level of a target molecule representative of expression of a gene encoding a protein selected from the group consisting of: RPL6; NCL; NPM; NAP1L1; SFRS2; FABP6; DDX5; and CBX3.

Methods in accordance with these embodiments may involve assaying a patient sample (or samples) for an elevated level of target molecules representative of expression of genes encoding two or three of the proteins HMGB1; PHB and CK18; in addition to assaying the patient sample (or samples) for the presence of target molecules representative of expression of genes encoding one or more markers selected from the group consisting of RPL6; NCL; NPM; NAP1L1; SFRS2; FABP6; DDX5; and CBX3, or alternatively such methods may involve assaying for an increase in the level of target molecules representative of expression of the genes encoding each of these proteins as required by the various aspects referred to herein.

The methods of the invention may be practiced using a wide variety of patient sample types. In general, the inventors believe that, except where the context of the disclosure requires otherwise, the methods in accordance with the various aspects of the invention may make use of any form of patient sample in which suitable target molecules representative of gene expression in a patient may be found. Merely by way of example, methods in accordance with the present invention may make use of patient samples is selected from the group consisting of:
body fluids—such as blood, serum, and urine;
body secretions—such as faeces; and
samples containing biological cells—such as tissue samples, including biopsy samples.

The skilled person will appreciate that the methods of the invention may be practiced using a range of different target molecules representative of expression of a gene of interest. Merely by way of example, a suitable target molecule representative of gene expression may comprise an RNA transcript translatable to yield a protein. mRNA of this sort will typically be found within biological cells in a patient sample, such as a biopsy or blood sample. Techniques by which mRNA may be collected, purified and amplified as necessary, are well known to those skilled in the art. In the event that it is wished to practice the method of the invention in an embodiment in which an RNA transcript is to be used as a target molecule, it will be appreciated that patient samples and assay techniques should be selected consistent with this aim.

Alternatively, a suitable target molecule may comprise the protein encoded by a gene, or precursors or variants produced on translation of the transcripts produced when the gene is expressed. Thus, in the event that a protein undergoes modification between first translation and its mature form, both the precursor and the mature protein may be used as suitable target molecules. As above, techniques by which protein target molecules may be preserved within a patient sample, thus facilitating its detection, will be well known to those skilled in the art.

Protein target molecules may be found associated with biological cells in patient samples. Additionally, or alternatively, patient samples may include extracellular protein, for example when the protein is secreted by cells in a soluble form or is released on breakdown of cells. Thus extracellular protein target molecules may be usefully investigated even in essentially acellular patient samples. It will be appreciated that samples containing biological cells may be preferred in the case that RNA transcripts are to be used as target molecules in methods in accordance with any of the aspects of the invention.

Examples of suitable patient samples and suitable assay techniques are described elsewhere in the present specification. It will be appreciated that the nature of a patient sample available may then determine the nature of target molecules and assay techniques that may be used in practicing a method of the invention.

The inventors have surprisingly found that different target molecule types may provide unforeseen advantages in the context of the different aspects of the invention. In particular, the Inventors have found that methods using protein target molecules may be particularly suitable for use in the diagnostic and prognostic aspects of the invention.

That said methods using mRNA target molecules provide a number of advantages, as discussed below, and their use may represent a preferred embodiment of the methods of the invention. By way of example, some advantages of the use of nucleic acid target molecules (such as mRNA) are as follows:
Assays for detection of nucleic acid target molecules (such as quantitative rtPCR or the like) tend to be cheaper than assays (such as ELISAs) for the detection of other target molecules (such as proteins).
Assays (such as PCR-based assays) for detection of nucleic acid target molecules are readily multiplexed, allowing investigation of expression of multiple targets within the same sample.
Stability of nucleic acid target molecules tends to be greater than for protein counterparts.
Processing of samples to obtain nucleic acid molecules is also typically simpler than processing required to obtain protein target molecules.
Numbers of nucleic acid target molecules within a sample can be amplified more readily (if required) than can protein target molecules.

The methods of the invention may make use of any appropriate assay by which the presence or elevated levels of a requisite target molecule may be detected. It will be appreciated that suitable assays may be determined with reference to the nature of the target molecule to be detected and/or the nature of the patient sample to be used.

Various suitable assay types will be known to those skilled in the art, including, but not limited to: enzyme linked immunosorbant assays (ELISA), including variants such as sandwich ELISAs; radioimmuno assays (RIA); immunocytochemistry labelling; immunohistochemistry labelling of a tissue sample; fluorescence activated cell sorting (FACS); chemiluminescence; reverse transcription PCR (rt PCR), and multiplex assays such as Luminex or proteomic MRM.

Serum samples represent particularly useful patient samples to be used when practising the methods of the invention. Serum samples can be readily collected from patients in relatively large volumes. Furthermore, the procedures for collection of serum samples are safe, commonplace, and not particularly invasive. Serum samples contain large amounts of the proteins identified as biomarkers in the disclosure of the present invention, and so allow good sensitivity of such methods.

The finding that the proteins identified in the full panel of biomarkers (or in specific sub-groups referred to elsewhere in the specification) are detectable in serum is surprising in that the prior art has previously suggested that the majority of these markers were not to be found in serum samples.

As set out elsewhere in the specification the use of methods in which protein marker molecules are investigated provides particular benefits in the context of methods in accordance with the aspects of the invention. It will thus be appreciated that certain samples represent beneficial patient samples to be used in preferred embodiments of such methods.

The inventors believe that analysis of the increase occurring across the sum of targets investigated in a method of the invention may be a useful manner in which the invention can be put into practice and suitably analysis performed. Analysis may be performed through relatively simple means, or may be undertaken using more complex algorithms. Examples of well known and freely available software that can be used for the analysis of results relating to expression of target molecules in the methods of the invention are described in the paragraphs below. Preferred methods by which analysis of the results achieved may be undertaken may give rise to further useful aspects and embodiments of the invention.

In a further aspect, the present invention provides kits for use in the methods described herein. Such kits may comprise binding partners capable of binding to a target molecule. In the case of a protein target molecule, such binding partners may comprise antibodies that bind specifically to the protein (e.g. an anti-NAP1L1 antibody). In the case of a nucleic acid target molecule the binding partner may comprise a nucleic acid complementary to the target molecule.

The invention will now be further described with reference to the following table, experimental results and figures in which:

Table 1 shows statistics compiled by Cancer Research UK comparing the approximate frequency and survival stage (at five years) for patients with colorectal cancer, as classified by Dukes' staging. These statistics illustrate that early identification of patients is important in increasing survival times, but that current procedures tend to identify patients with colorectal cancer at relatively late stages of disease progression, with adverse impact on patient survival.

Table 2 shows a summary of descriptions and properties of a number of potential biomarkers investigated by the Inventors.

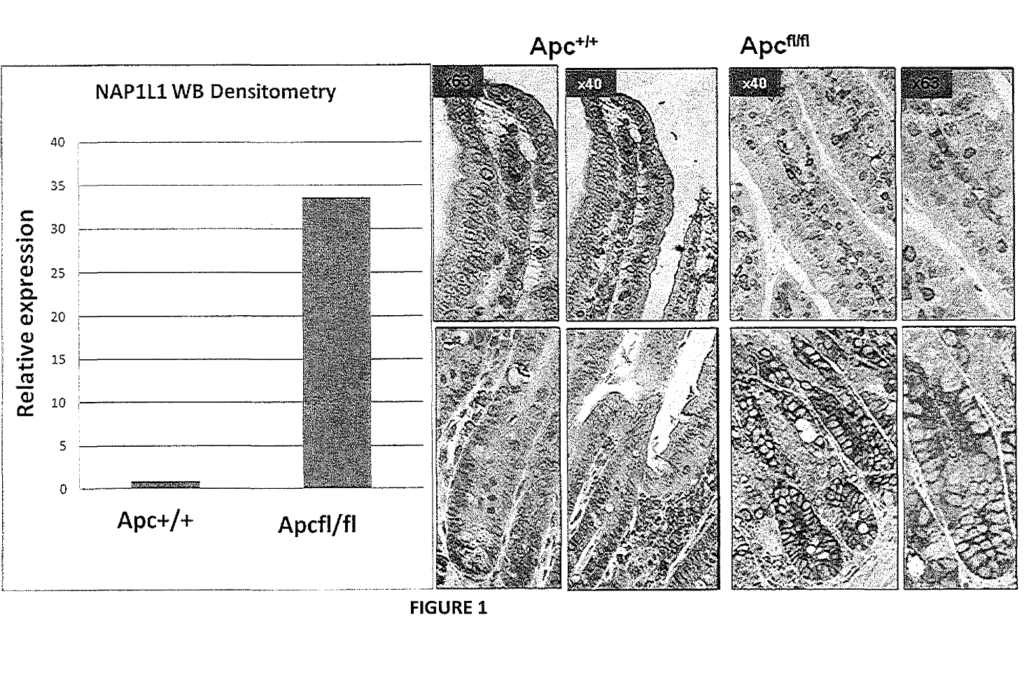

FIG. 1 illustrates immunolabelling of NAP1L1 in tissue samples from a mouse model of colorectal cancer, and results of Western blotting for this protein in cancer model and control samples.

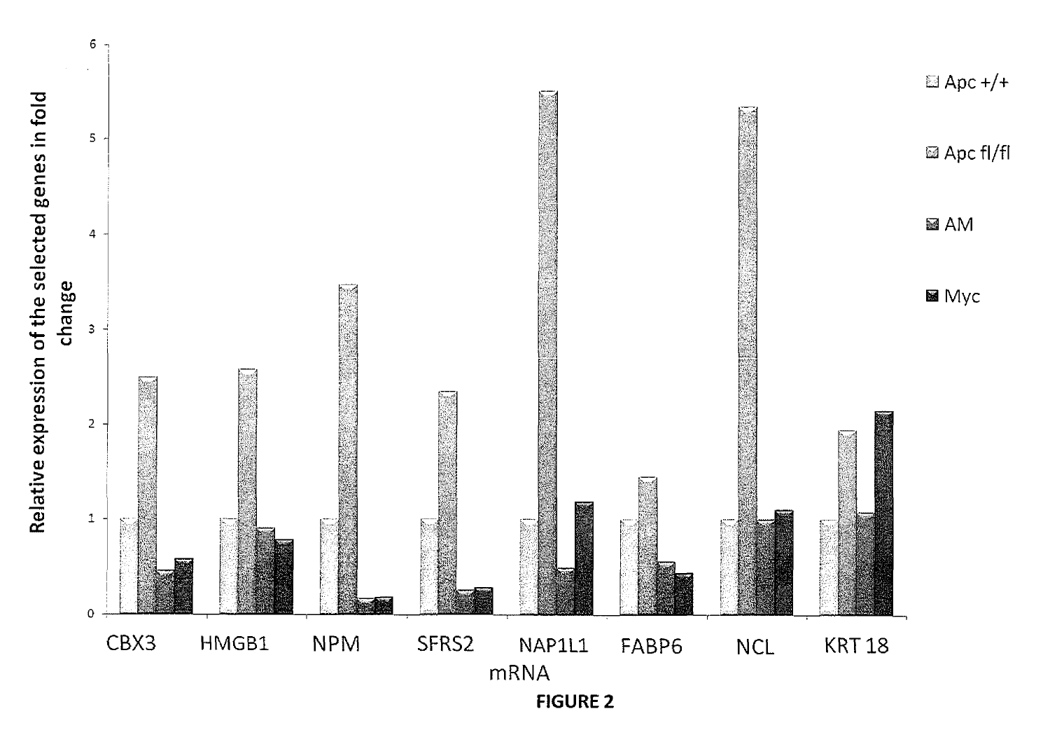

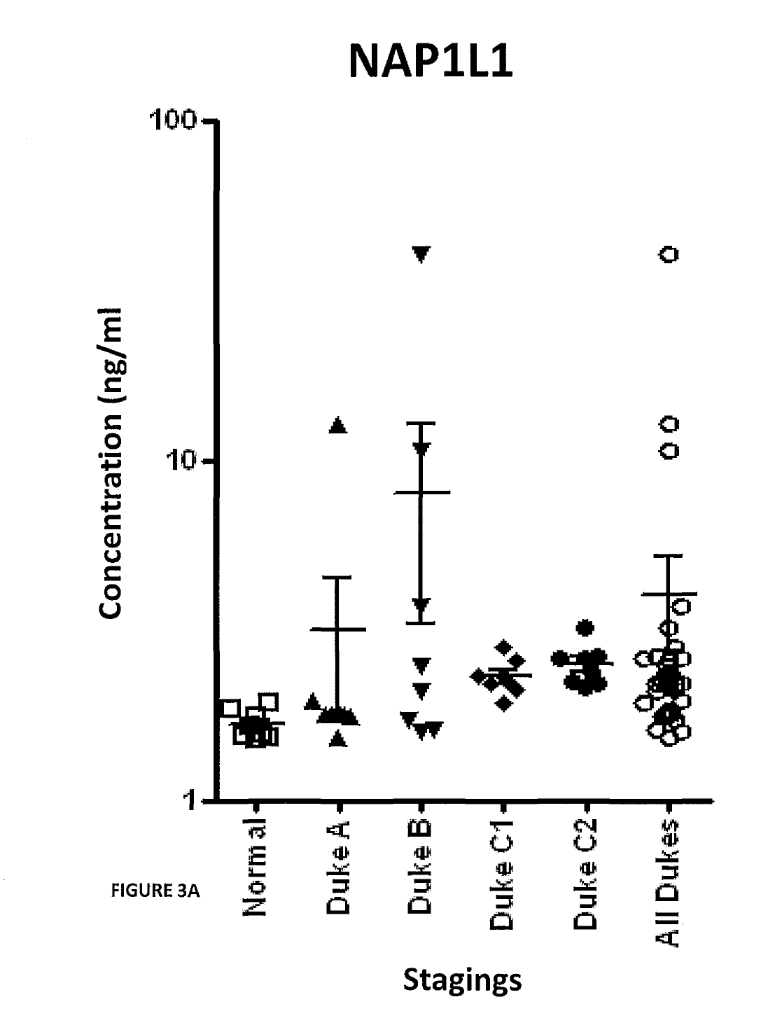

FIG. 2 illustrates relative expression of eight selected genes in various mouse models.

FIG. 3 illustrates the results of sandwich ELISAs for NAP1L1 in human control and cancer samples (Panel A), receiver operating characteristic statistical analysis of these results (Panel B), and fold changes in NAP1L1 expression between normal and cancer samples as determined by PCR analysis

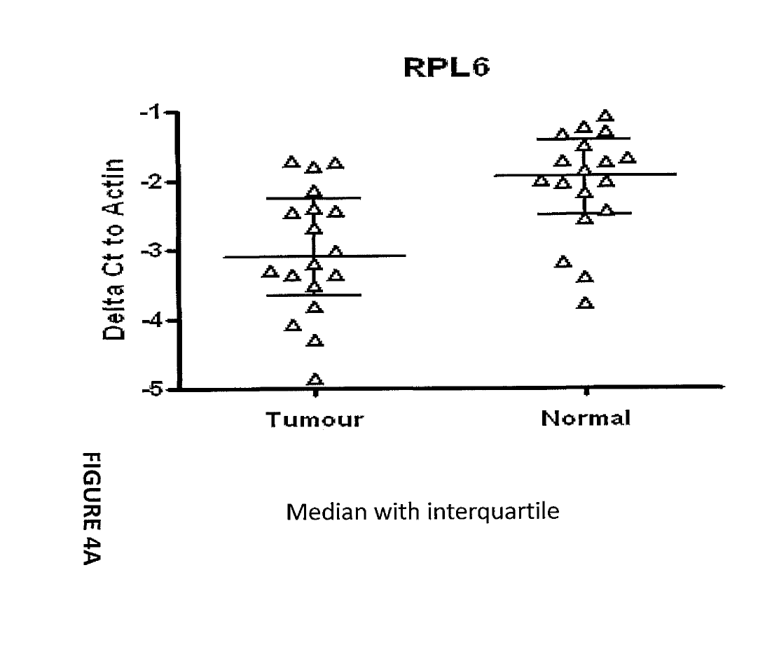

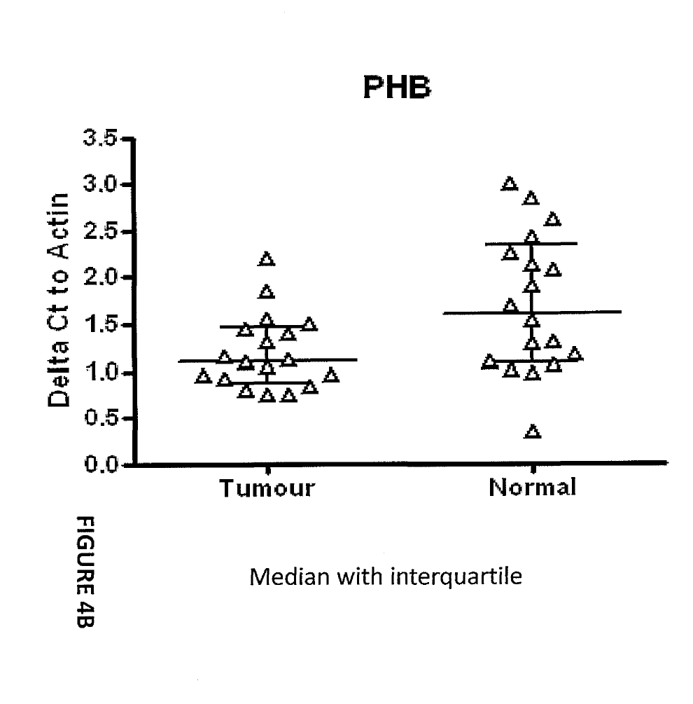

FIG. 4 illustrates ROC analysis of changes in expression of RPL6 (Panel A), PHB (Panel B), CK18 (Panel C), HMGB1 (Panel D), FABP6 (Panel E), and NPM (Panel F) in tumour and control samples as determined by PCR analysis.

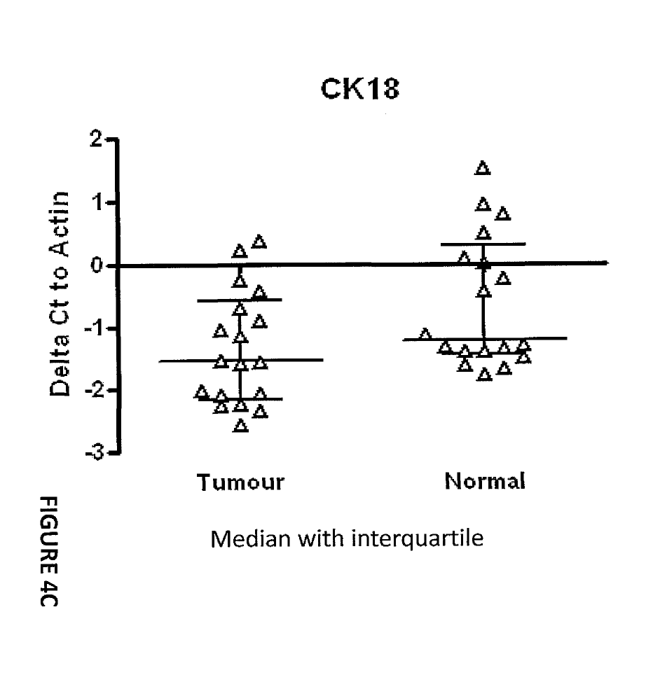

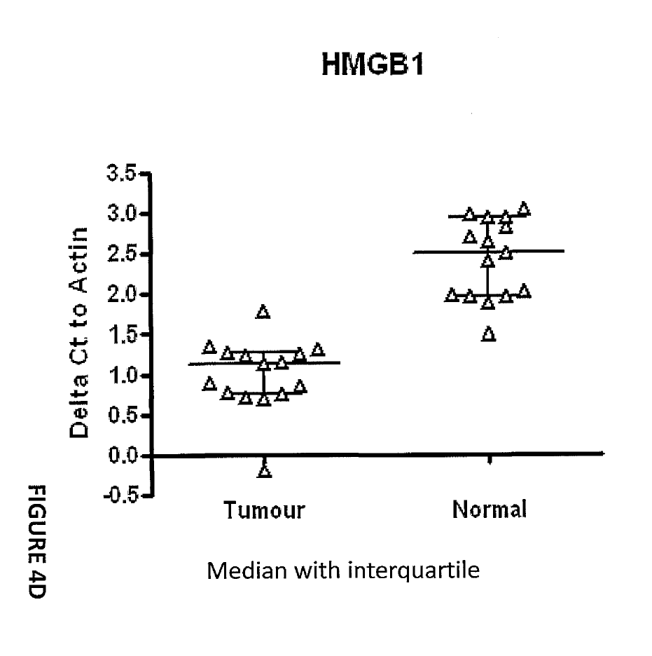

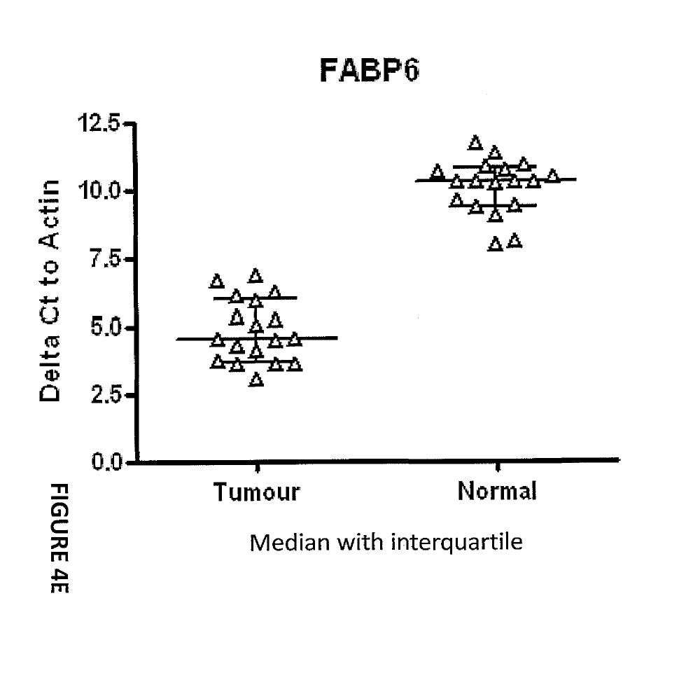

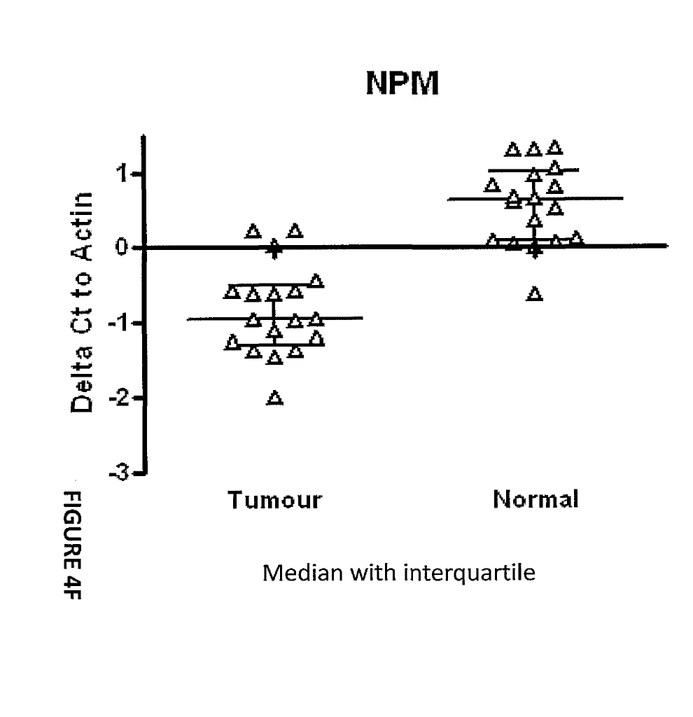

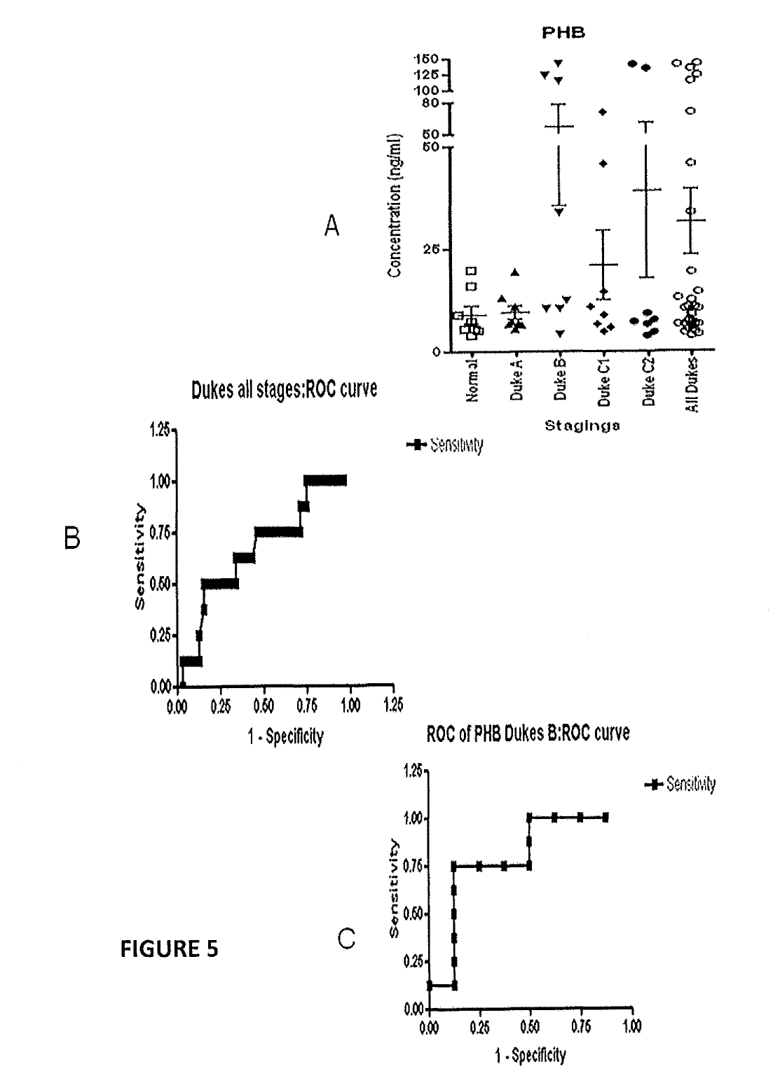

FIG. 5 illustrates differences in PHB expression between control and tumour samples, as assessed by ELISA, and statistical analysis of these differences.

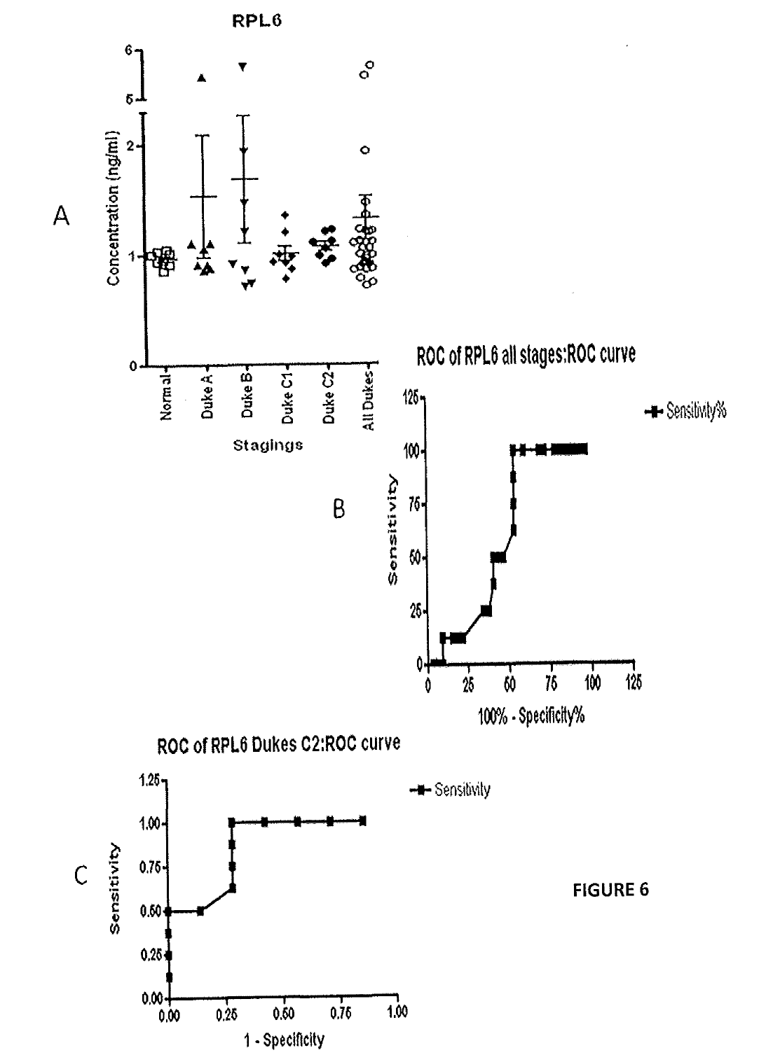

FIG. 6 illustrates differences in RPL6 expression between control and tumour samples, as assessed by ELISA, and statistical analysis of these differences.

FIG. 7 illustrates changes in NAP1L1 expression between control and cancer model mice, as determined by PCR, with or without amplification of target molecule numbers.

FIG. 8 illustrates changes in PHB expression between control and cancer model mice, as determined by PCR, with or without amplification of target molecule numbers.

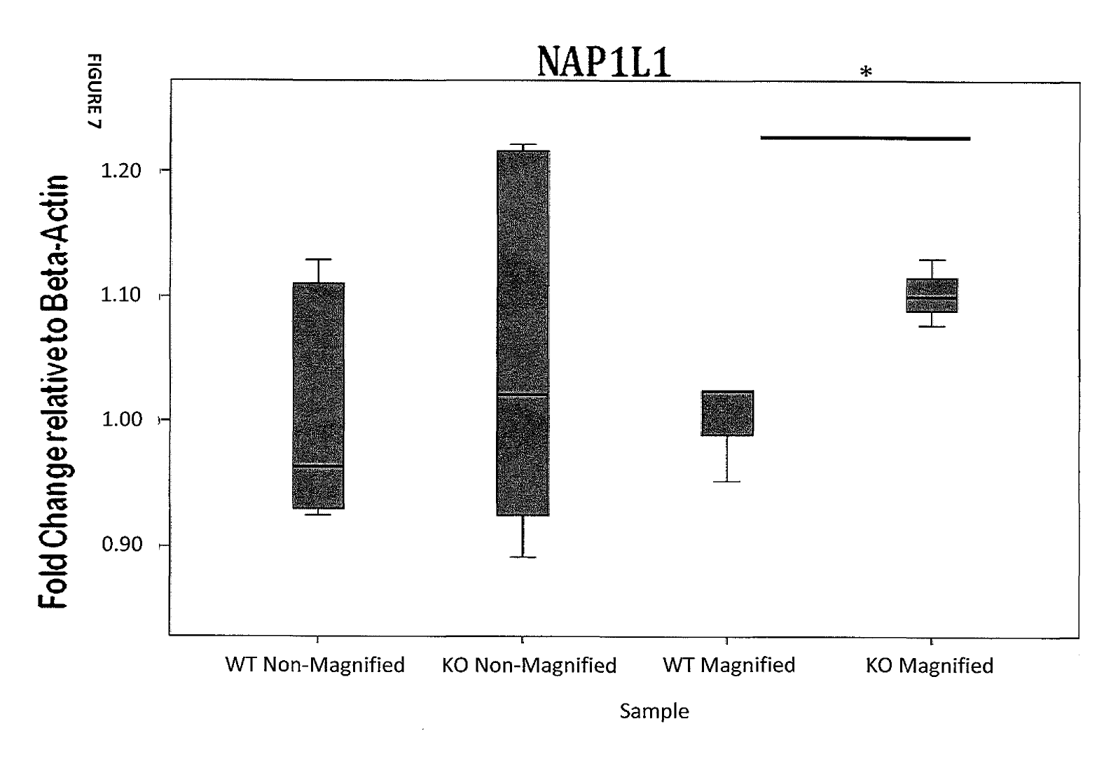

FIG. 9 illustrates changes in CK18 expression between control and cancer model mice, as determined by PCR, with or without amplification of target molecule numbers.

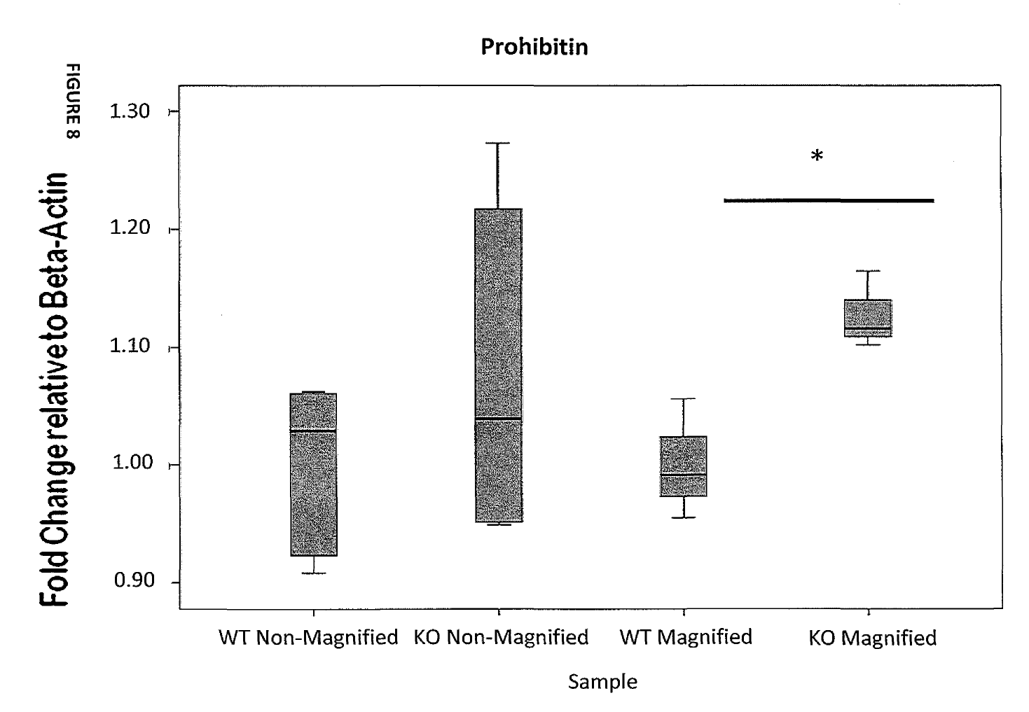

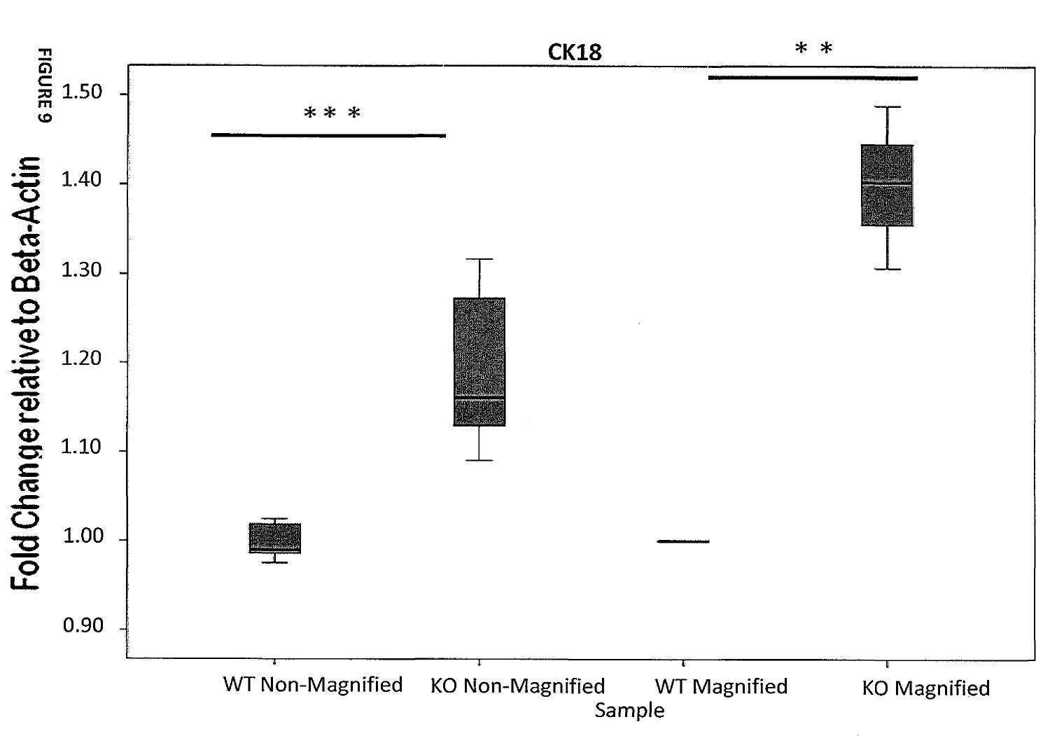

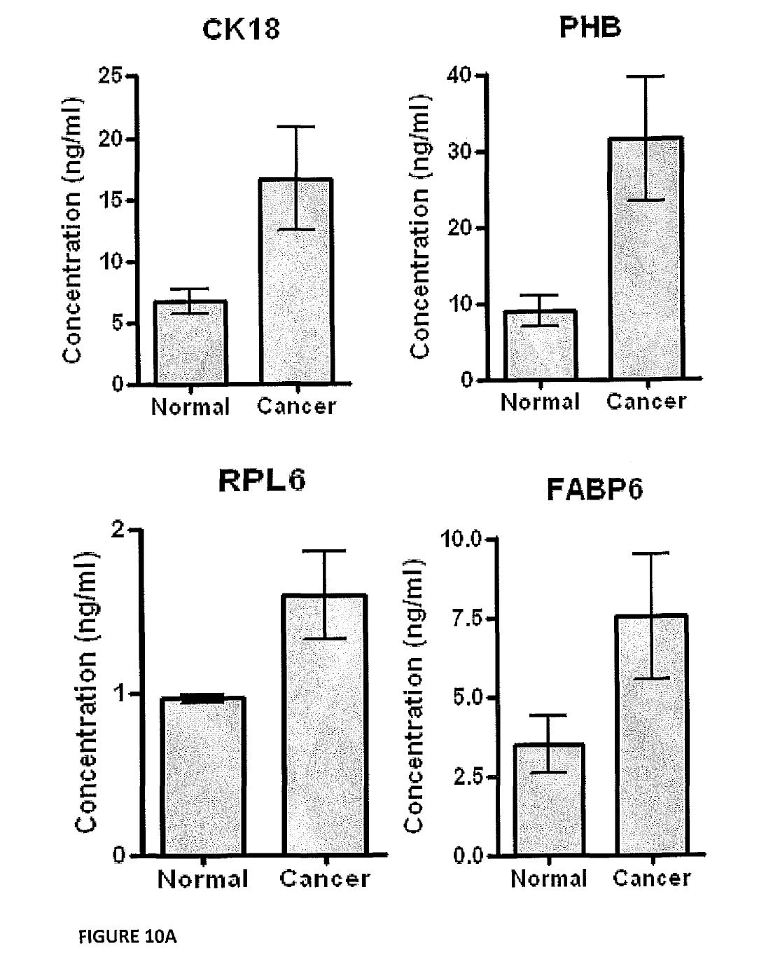

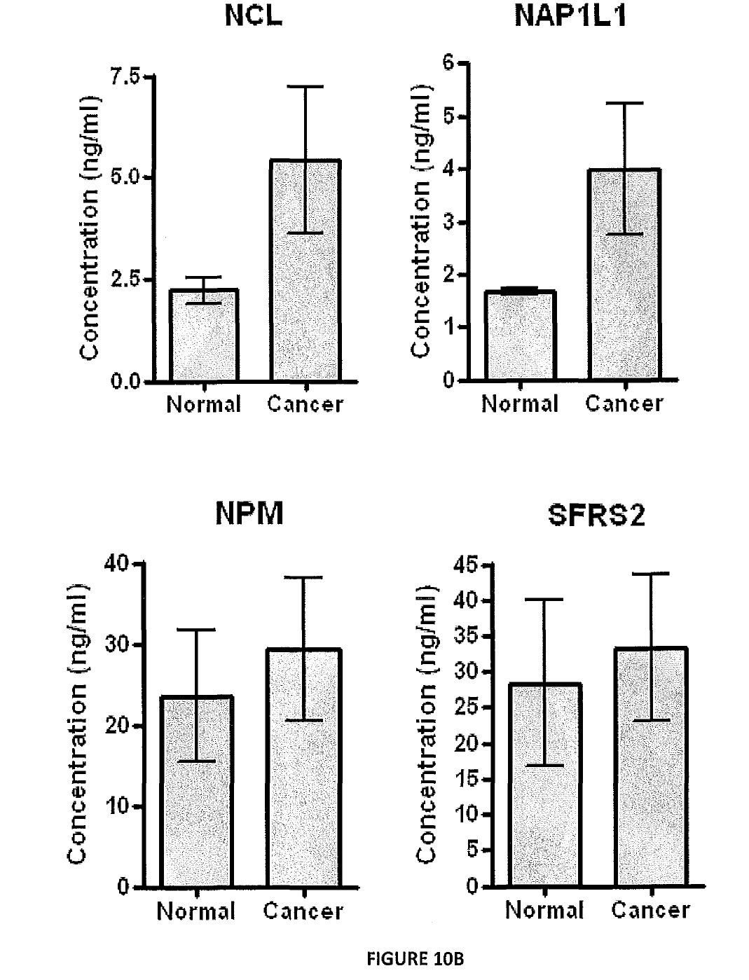

FIG. 10 illustrates increased expression of biomarkers in cancer samples when compared to the level of expression occurring in a control sample.

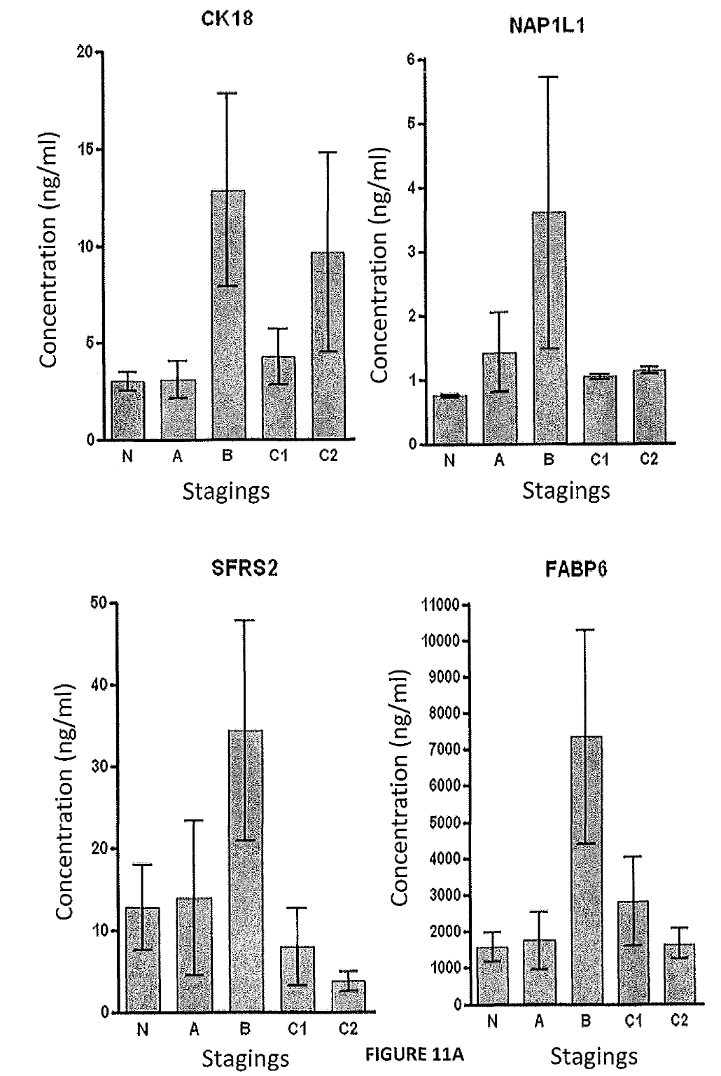

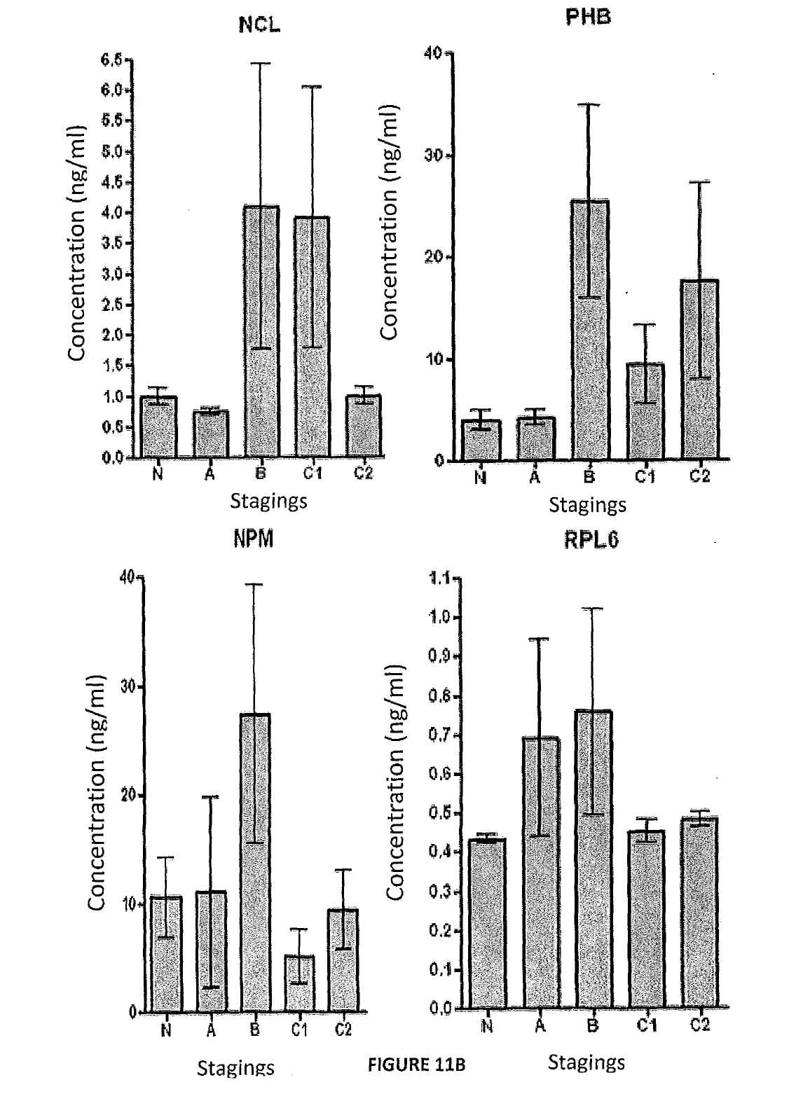

FIG. 11 illustrates expression of biomarkers in patient samples from colon cancer patients at various stages of the disease (assessed using Dukes staging from A to C2) compared with serum samples from normal patients without cancer.

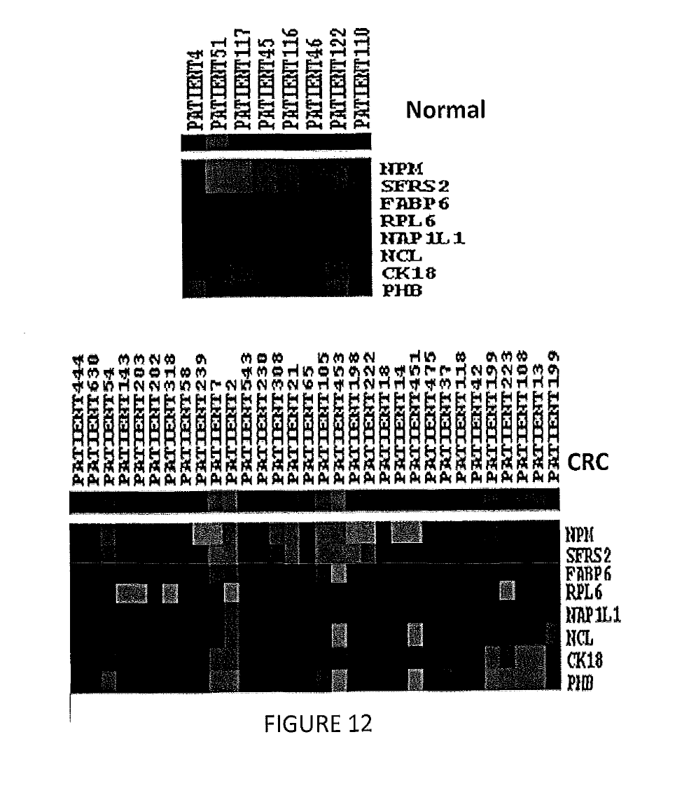

FIG. 12 illustrates a comparison between expression of the biomarkers PHB, CK18, NCL, NAP1L1, RPL6, FABP6, SFRS2, or NPM and the length of patient survival.

FIG. 13 illustrates the results of analysis of expression of PHB plotted against cancer patient survival.

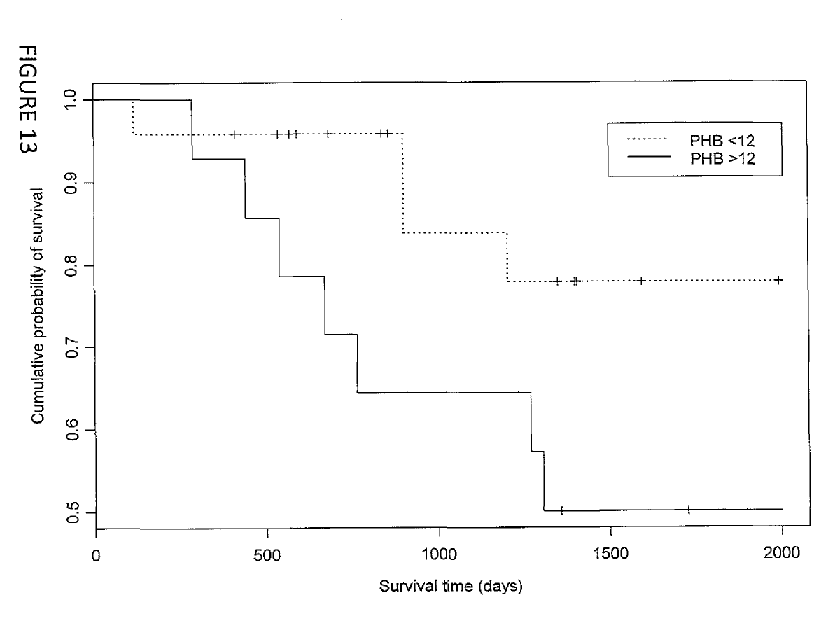

FIG. 14 illustrates the results of analysis of expression of CK18 plotted against cancer patient survival.

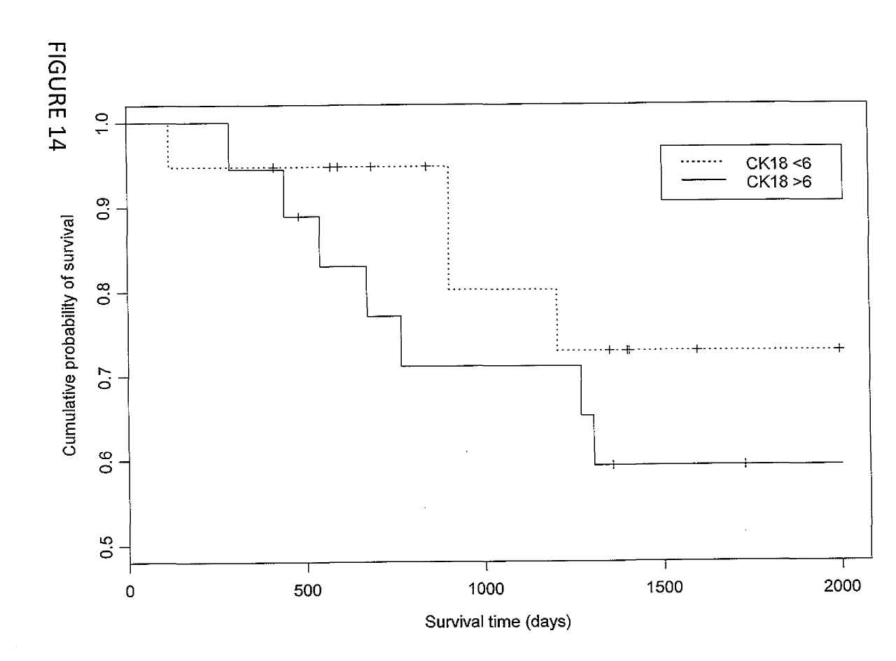

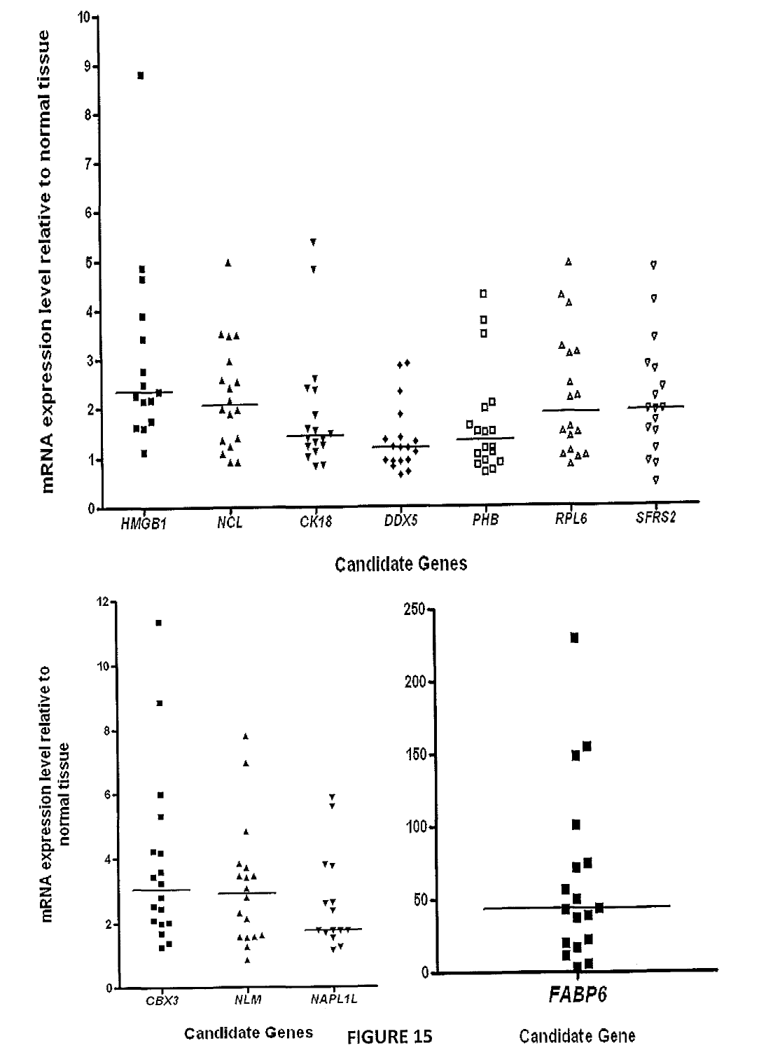

FIG. 15 illustrates characteristic increases in expression of the biomarkers investigated in assays in which RNA is used as a target molecule representative of gene expression.

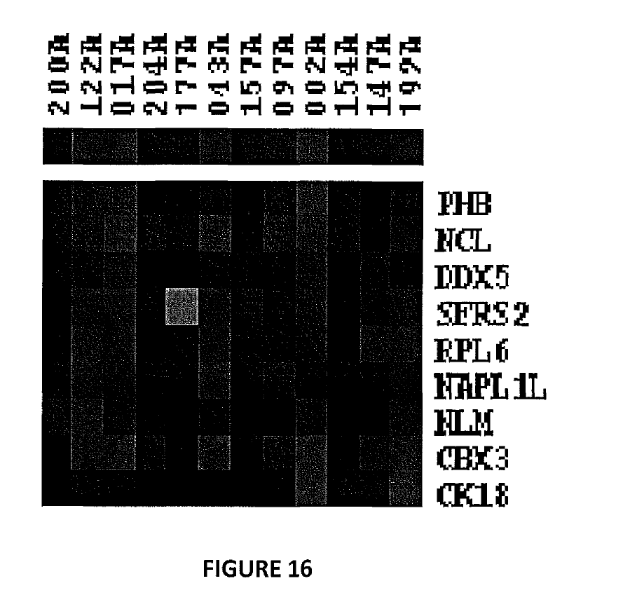

FIG. 16 illustrates the results of a comparison of biomarker expression (assessed via investigation of RNA target molecules) with patient survival.

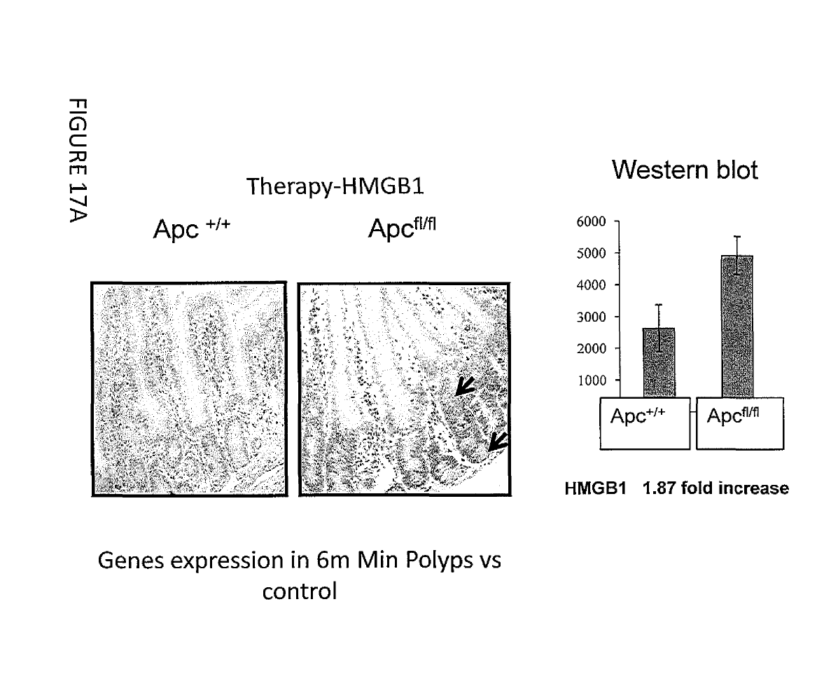

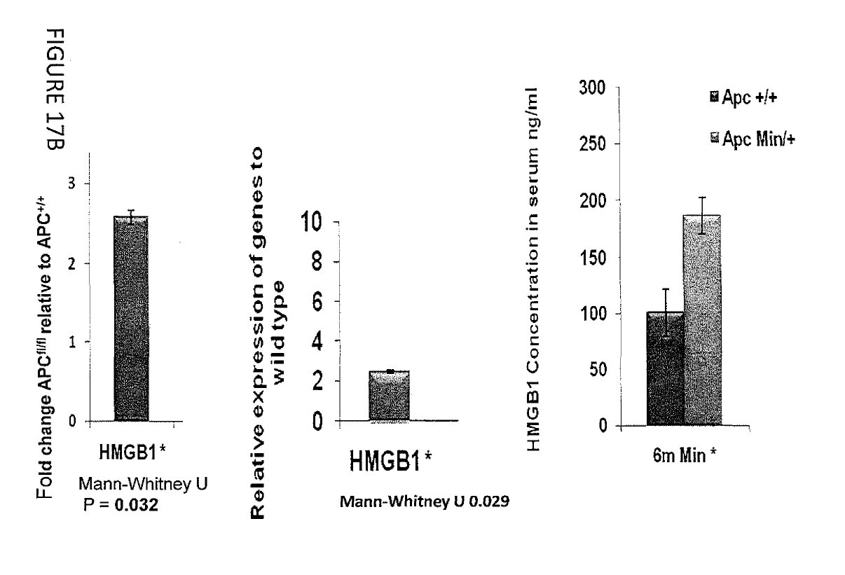

FIG. 17 illustrates the results of an investigation into gene expression within the intestinal epithelia of APC^{fl/fl} mice, which illustrates that there is a marked increase in HMGB1 expression.

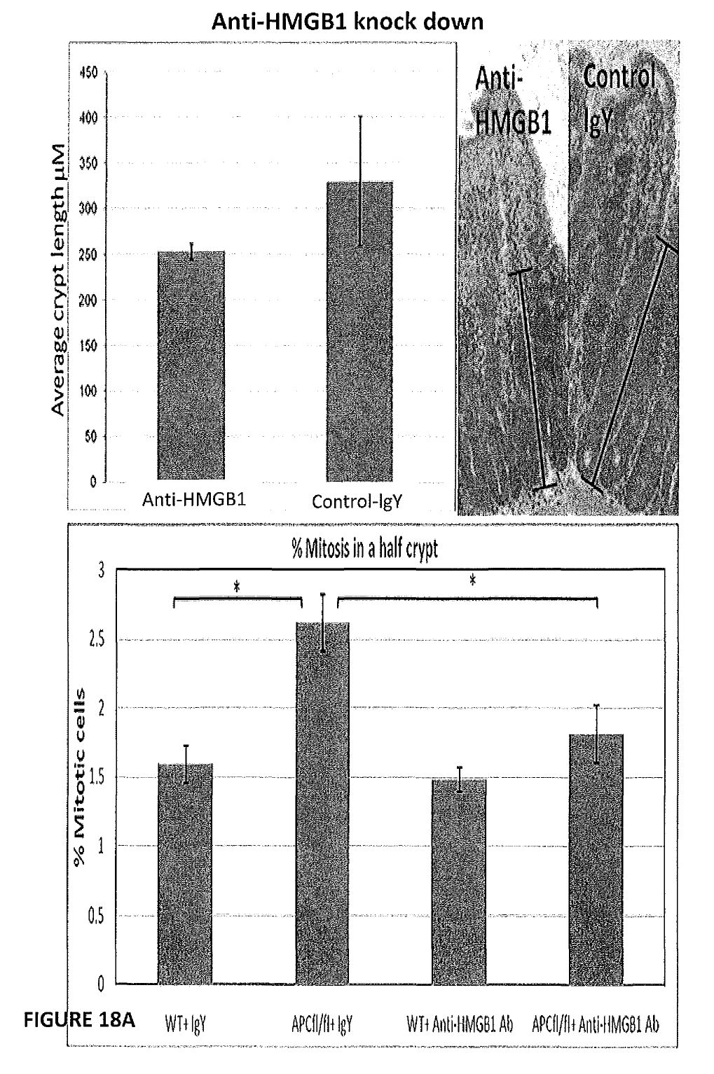

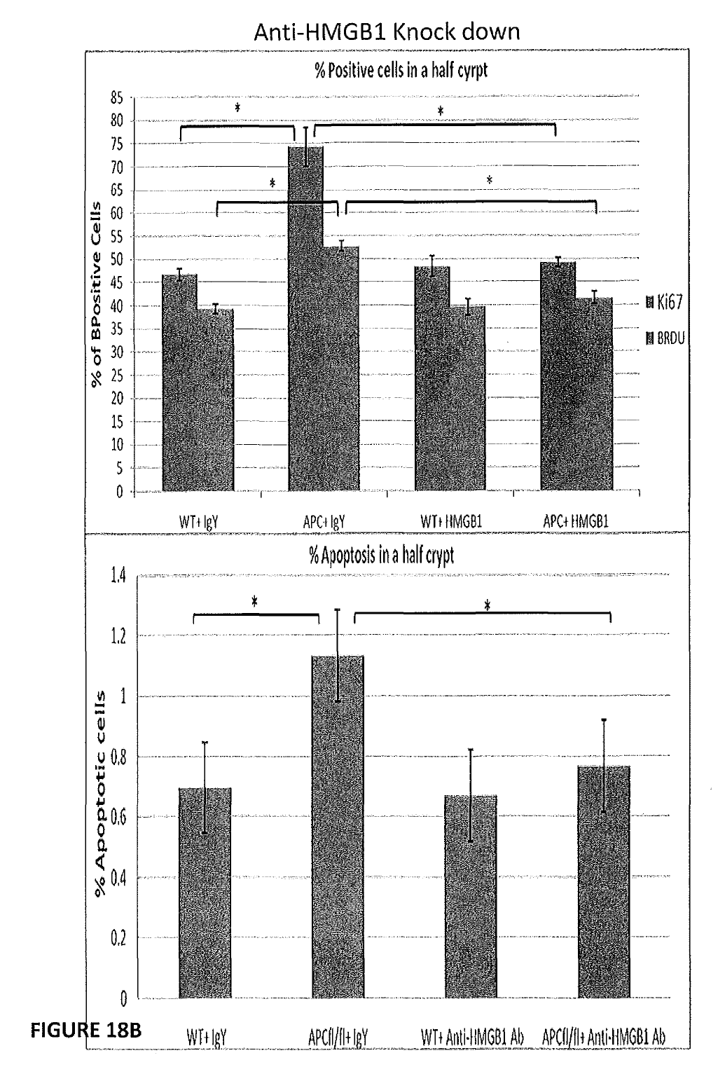

FIG. 18 illustrates that treatment of APC^{fl/fl} mice with an anti-HMGB1 antibody, capable of inhibiting the cytokine effect of HMGB1, is able to reduce crypt length, and also to restore levels of cell division and apoptosis to near normal levels found in wild type (WT) mice with or without control antibody.

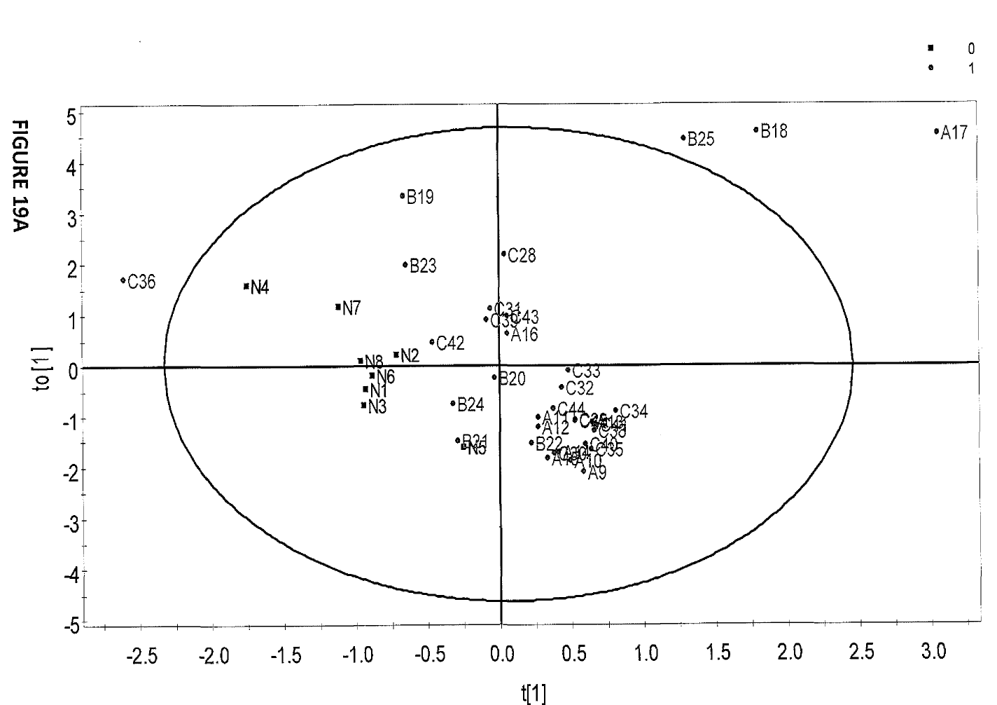

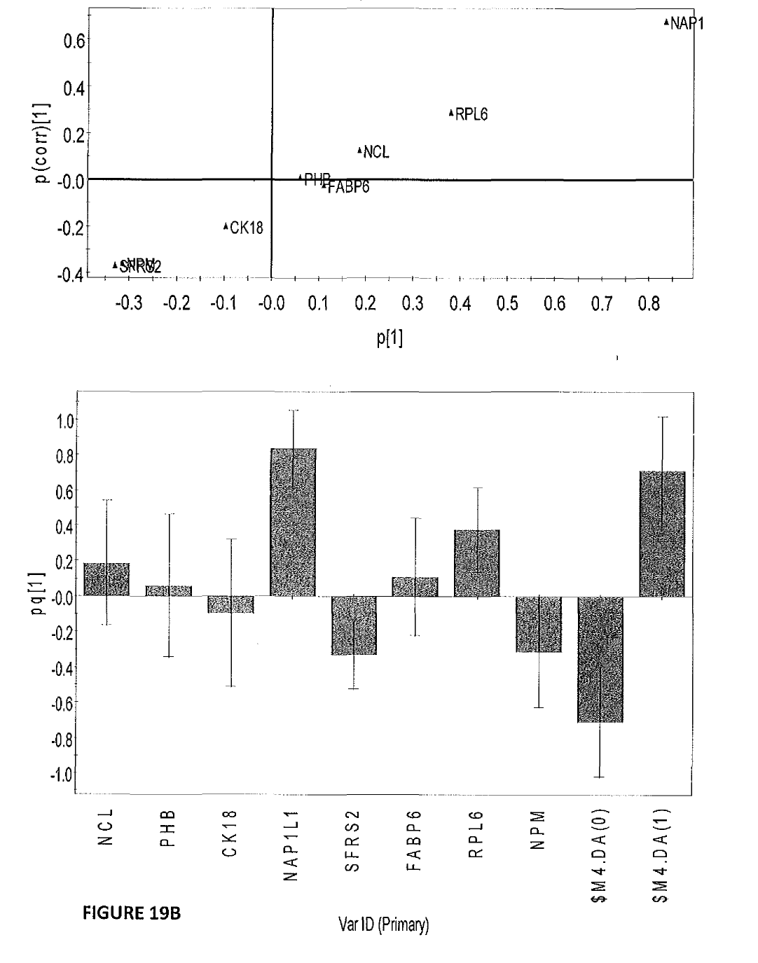

FIG. 19 illustrates the results of principal component analysis (PCA) showing the relative contribution of a range of biomarkers (NCL, PHB, CK18, NAP1L1, SFRS2, FABP6, RPL6, and NPM) to the ability to distinguish between control samples, and samples derived from cancer patients (without reference to cancer staging).

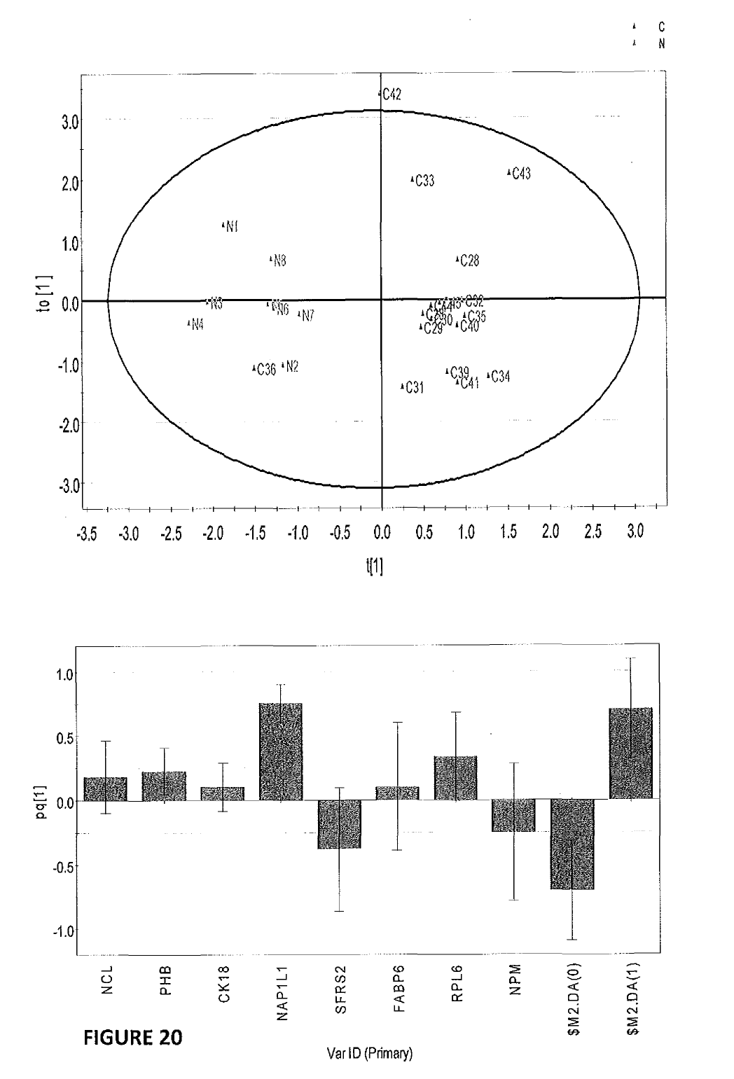

FIG. 20 illustrates the results of principal component analysis (PCA) showing the relative contribution of a range of biomarkers (NCL, PHB, CK18, NAP1L1, SFRS2, FABP6, RPL6, and NPM) to the ability to distinguish between control samples, and samples derived from cancer patients staged as Duke's C.

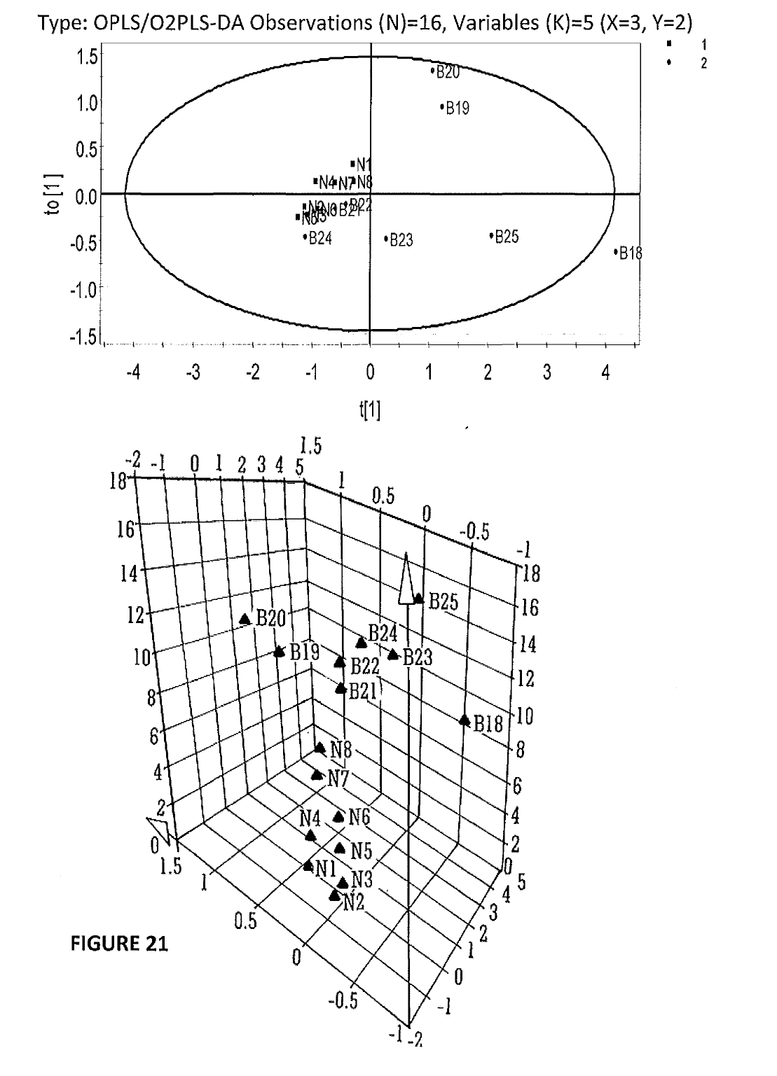

FIG. 21 illustrates the results of PCA based on expression of RPL6 and NAP1L1 in control samples and samples from Duke's stage B colorectal cancer.

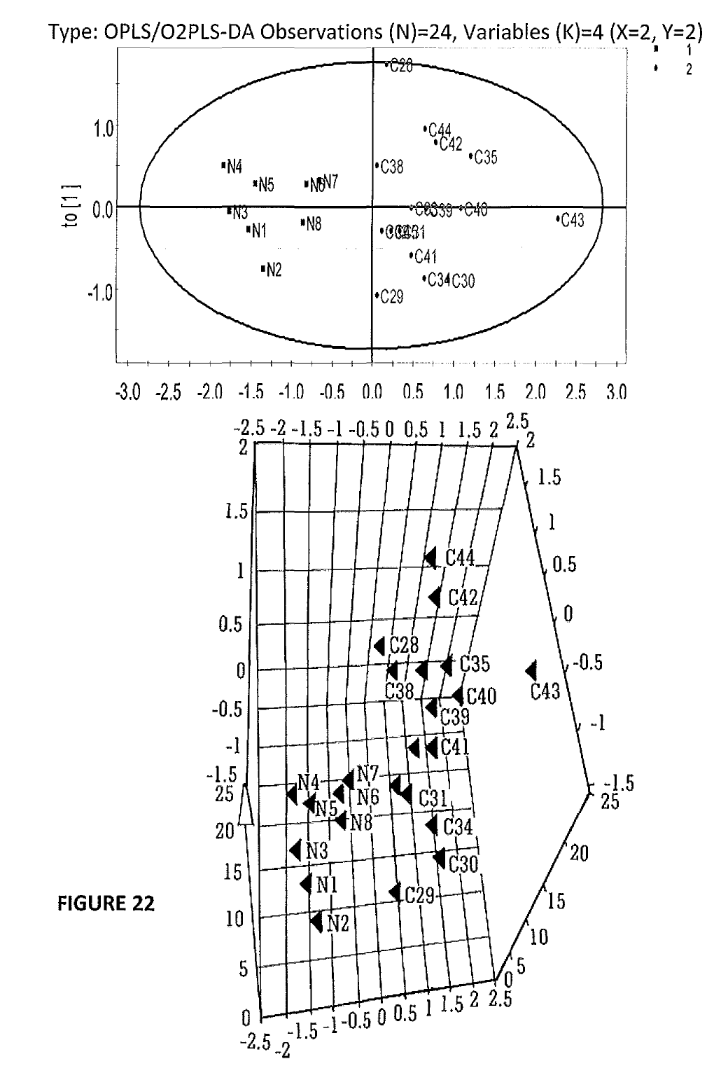

FIG. 22 illustrates the results of PCA based on expression of RPL6 and NAP1L1 in control samples and samples from Duke's stage C colorectal cancer.

FIG. LEGENDS

```
FIG. 1
Candidate protein, NAP1L1 in Apc^{fl/fl} and Apc^{+/+} mice (colon cancer model)
    • Nucleosome assembly protein1 like 1
    • Modulation of chromatin formation
    • DNA replication
    • Regulation of cell proliferation
    • Position in cells
Apc^{fl/fl} and wild type control small intestinal sections, anti-NAP1L1 Rb polyclonal antibody.
Brown staining equates with over expression
FIG. 2
Relative expression of 8 selected genes (CBX3, HMGB1, NPM, SFRS2, NAP1L1, FABP6,
NCL, and KRT18) using mRNA extracted from ECE samples from Apc^{+/+}, Apc^{fl/fl}, Apc^{fl/fl}
Myc^{fl/fl} (AM), and Apc^{+/+} Myc^{fl/fl} (Myc) mouse models (4 or 5 mice in each group) showing the
fold change in mRNA expression for each gene relative to the Apc^{+/+} wild type.
Demonstrating message over expression in a mouse colon cancer model and that this
expression is probably regulated through c-myc.
FIG. 3
Detection of NAP1L1 in human serum by sandwich ELISA,
protein concentrations in human serum samples.
A) Data are presented for normal subjects and CRC patients with Dukes' A, Dukes' B,
Dukes' C1, Dukes' C2 tumours and all CRC Dukes stages combined. Dot-plot shows
NAPL1L concentrations in individual patients with the horizontal line indicating the mean.
There are 8 samples in each normal or Dukes' stage group.
    • Horizontal line with arrow bars show the mean with SEM
Log 10 Y-axis
FIG. 3B
ROC analysis of serum NAP1L1 protein concentration
ROC of NAP1L1 Dukes A: ROC curve
    Area under the ROC curve
```

| | |
|---|---|
| Area | 0.7344 |
| Std. Error | 0.1342 |
| 95% confidence interval | 0.4712 to 0.9975 |
| P value | 0.1152 |
| Data | |
| Control | 8 |
| Patient | 8 |

ROC of NAP1L1 Dukes B: ROC curve
Area under the ROC curve

| | |
|---|---|
| Area | 0.8281 |
| Std. Error | 0.1056 |
| 95% confidence interval | 0.6212 to 1.035 |
| P value | 0.02747 |
| Data | |
| Control | 8 |
| Patient | 8 |

ROC of NAP1L1 Dukes C1: ROC curve
Area under the ROC curve

| | |
|---|---|
| Area | 1.000 |
| Std. Error | 0.0 |
| 95% confidence interval | 1.000 to 1.000 |
| P value | 0.0007834 |
| Data | |
| Control | 8 |
| Patient | 8 |

ROC of NAP1L1 Dukes C2: ROC curve
Area under the ROC curve

| | |
|---|---|
| Area | 1.000 |
| Std. Error | 0.0 |
| 95% confidence interval | 1.000 to 1.000 |
| P value | 0.0007834 |
| Data | |
| Control | 8 |
| Patient | 8 |

ROC of NAP1L1 all stages: ROC curve
Area under the ROC curve

| | |
|---|---|
| Area | 0.8906 |
| Std. Error | 0.05365 |
| 95% confidence interval | 0.7854 to 0.9958 |
| P value | 0.0007271 |
| Data | |
| Control | 8 |
| Patient | 32 |
| FIG. 3C | |

| | |
|---|---|
| Column A | Tumour |
| vs | vs |
| Column B | Normal |
| Mann Whitney test | |
| P value | P < 0.0001 |
| Exact or approximate P value? | Gaussian Approximation |
| P value summary | *** |
| Are medians signif. different? (P < 0.05) | Yes |
| One- or two-tailed P value? | Two-tailed |
| Sum of ranks in column A, B | 184, 482 |
| Mann-Whitney U | 13.00 |
| FIG. 4A | |
| Column A | Tumour |
| vs | vs |
| Column B | Normal |
| Mann Whitney test | |
| P value | 0.0025 |
| Exact or approximate P value? | Gaussian Approximation |
| P value summary | ** |
| Are medians signif. different? (P < 0.05) | Yes |
| One- or two-tailed P value? | Two-tailed |
| Sum of ranks in column A, B | 237, 429 |
| Mann-Whitney U | 66.00 |
| FIG. 4B | |
| Column A | Tumour |
| vs | vs |
| Column B | Normal |
| Mann Whitney test | |
| P value | 0.0201 |
| Exact or approximate P value? | Gaussian Approximation |
| P value summary | * |
| Are medians signif. different? (P < 0.05) | Yes |
| One- or two-tailed P value? | Two-tailed |
| Sum of ranks in column A, B | 259, 407 |
| Mann-Whitney U | 88.00 |
| FIG. 4C | |
| Column A | Tumour |
| vs | vs |
| Column B | Normal |
| Mann Whitney test | |
| P value | 0.0413 |
| Exact or approximate P value? | Gaussian Approximation |
| P value summary | * |
| Are medians signif. different? (P < 0.05) | Yes |
| One- or two-tailed P value? | Two-tailed |
| Sum of ranks in column A, B | 268, 398 |
| Mann-Whitney U | 97.00 |
| FIG. 4D | |
| Column A | Tumour |
| vs | vs |
| Column B | Normal |
| Mann Whitney test | |
| P value | P < 0.0001 |
| Exact or approximate P value? | Gaussian Approximation |
| P value summary | *** |
| Are medians signif. different? (P < 0.05) | Yes |
| One- or two-tailed P value? | Two-tailed |
| Sum of ranks in column A, B | 121, 344 |
| Mann-Whitney U | 1.000 |
| FIG. 4E | |
| Column A | Tumour |
| vs | vs |
| Column B | Normal |
| Mann Whitney test | |
| P value | P < 0.0001 |
| Exact or approximate P value? | Gaussian Approximation |
| P value summary | *** |
| Are medians signif. different? (P < 0.05) | Yes |
| One- or two-tailed P value? | Two-tailed |
| Sum of ranks in column A, B | 171, 495 |
| Mann-Whitney U | 0.0000 |
| FIG. 4F | |
| Column A | Tumour |
| vs | vs |
| Column B | Normal |
| Mann Whitney test | |
| P value | P < 0.0001 |
| Exact or approximate P value? | Gaussian Approximation |
| P value summary | *** |
| Are medians signif. different? (P < 0.05) | Yes |
| One- or two-tailed P value? | Two-tailed |
| Sum of ranks in column A, B | 188, 478 |
| Mann-Whitney U | 17.00 |

FIG. 10A and FIG. 10B
Detection in the serum of colon cancer patients verses normal (ELISA)
FIG. 11A and FIG. 11B
Detection in the serum of colon cancer patients at various stages of the disease (ELISA) 8
patient samples from each stage (N = normal, A-C2 - Dukes staging)
FIG. 12
General
Red CK18 & PHB bad prognosis-poor survival (unless SFRS2 strongly up as well)
Strong red SFRS2 good survival marker 5 yrs + or from diagnosis and still alive
FIG. 13
Cancer Survival Analysis with reference to PHB expression

| | | |
|---|---|---|
| Wald test = | 5.42 on 1 df, | p = 0.0199 |
| Score (logrank) test = | 6.32 on 1 df, | p = 0.012 |

FIG. 14
Cancer Survival Analysis with reference to CK18 expression

| | | |
|---|---|---|
| Wald test = | 5.82 on 1 df, | p = 0.0159 |
| Score (logrank) test = | 6.8 on 1 df, | p = 0.00914 |

FIG. 15
Relative expression tumour matched to adjacent normal tissue
All statistically significant by Wilcoxon Signed Rank Test
FIG. 16
Relative expression tumour matched to adjacent normal tissue
Red = 4/4 dead before 5 yrs (3.75 yr survival*)
no red/black = 2/6 survived 5 years + (5.5 yr survival*)
*Extremely rough calculation based on no death = 10 yr survival
192A died non CRC related (two weeks after 1stdiagnosis)
FIG. 19A
Normal verses all CRC
FIG. 19B
S-plot (contribution)
FIG. 20
Normal verses grade C's only
FIG. 21
Cancer data
Normals vs B's
M12 - N vs Bs

| | | | | | | | | |
|---|---|---|---|---|---|---|---|---|
| A | R2X | R2X(cum) | Eigenvalue | R2Y | R2Y(cum) | Q2 | Q2(cum) | |
| Model | | 0.988 | | | 0.335 | | −0.242 | |
| 0 | Cent. | | | Cent. | | | | |
| 1 | 0.729 | 0.988 | 2.19 | 0.335 | 0.335 | −0.242 | −0.242 | R1 |
| Orthogonal | | 0.259 | | | 0 | | | |
| 1 | 0.259 | 0.259 | 0.776 | 0 | 0 | | | NS |

FIG. 22
Normals vs C's
NAP1L1 and RPL6 only
M8 - M7 NAP1 and RPL6 only

| | | | | | | | | |
|---|---|---|---|---|---|---|---|---|
| A | R2X | R2X(cum) | Eigenvalue | R2Y | R2Y(cum) | Q2 | Q2(cum) | |
| Model | 1 | | | | 0.76 | | 0.683 | |
| 0 | Cent. | | | Cent. | | | | |
| 1 | 0.575 | 1 | 1.15 | 0.76 | 0.76 | 0.683 | 0.683 | R1 |
| Orthogonal | | 0.418 | | | | 0 | | |
| 1 | 0.418 | 0.418 | 0.838 | 0 | 0 | | | R1 |

### Experimental Results

### 1 NAP1L1 as a Biomarker for Cancer, or Precancerous Conditions, of the Colon

The utility of NAP1L1 as a biomarker for cancer of the colon, or precancerous conditions of the colon, was clearly illustrated by the following studies.

1.1 Immunolabelling and Localisation of NAP1L1 Protein in a Mouse Model of Colon cancer

Colonic expression of NAP1L1 was investigated in tissue samples taken from the Apc^{fl/fl} model of colon cancer (in which Apc is acutely deleted in epithelium of the colon, causing cancerous changes). Tissue samples were labelled with a rabbit anti-mouse polyclonal antibody specific for NAP1L1, and binding of the primary antibody visualised using a chromogenic enzyme. The results of this labelling are shown in FIG. 1, in which samples from Apcfl/fl mice (in which cancerous changes have taken place) are compared with matched samples from control Apc+/+ animals. The presence of NAP1L1 protein is indicated by generation of a dark brown colour, and cell nuclei are visualised by blue counter-staining.

It can be seen that expression of NAP1L1 is elevated in cancerous tissue samples as compared to controls, and that the NAP1L1 protein has an extranuclear location.

### 1.2 Western Blotting Analysis of NAP1L1 Expression in a Mouse Model of Colorectal Cancer

The difference in NAP1L1 expression between normal Apc^{+/+} mice and the Apc^{fl/fl} mice of the colon cancer model was further illustrated by densitometric comparison of the results of Western blots conducted on samples produced from each group of animals. As can be seen in FIG. 1, NAP1L1 expression in Apc^{fl/fl} mice was approximately 34 times the level occurring in normal Apc^{+/+} mice.

### 1.3 Investigation of Control of NAP1L1 Expression in a Mouse Model of Colorectal Cancer

The regulation of NAP1L1 expression in this mouse model of colon cancer was further investigated by comparing relative expression of NAP1L1 (among other genes) in mice in which Apc and/or c-myc expression was manipulated. As shown in FIG. 2, expression of the biomarker genes was investigated in four different varieties of mice (left to right for each gene investigated: Apc^{+/+}; Apc^{fl/fl}; “AM” in which both Apc and Myc were conditionally deleted; and “Myc” in which Apc remains unaffected, but Myc is conditionally deleted), and the results indicate that NAP1L1 expression in the mouse model of colon cancer is most likely regulated via c-myc.

### 1.4 Elevated Expression of NAP1L1 in Human Colorectal Cancer Samples, as Compared to Control Tissue

The results observed in the mouse model of colon cancer were confirmed in a study conducted using human colorectal cancer tumour samples. The presence of protein or nucleic acid target molecules representative of expression of NAP1L1 in these samples were respectively investigated via sandwich ELISA and quantitative real time PCR analysis.

Sandwich ELISAs for PHB or RPL6 were undertaken using sandwich enzyme immunoassays purchased from Uscn Life Science Inc. Assays were performed following the manufacturer's instructions. Briefly, 100 μl samples, serum and dilution buffer, and protein standards were incubated in pre-coated 96-well plates for 2 hours at 37° C. Then the liquid was removed and 100 μl of detection reagent A was added and incubated for 1 hour at 37° C. After 3 washes with 350 μl of 1× washing solution, 100 ul of detection reagent B was added and incubated for 30 minutes at 37° C. After 5 washes, 90 μl of substrate solution was added to each well. Following incubation for 15-25 minutes at 37° C., 50 ul of stop solution was added to each well. The optical density (O.D.) was immediately measured at 450 nm using a microplate reader (Dynex ELISA plate reader). O.D. values were plotted against the known concentration of each standard to generate a standard curve, from which the concentration of each sample was calculated in ng/ml.

The results of these investigations are shown in Panels A and B of FIG. 3.

FIG. 3A shows the result of sandwich ELISAs to detect expressed NAP1L1 protein in control samples (labelled “normal”) and in samples taken from patients whose colorectal cancer had been staged as Dukes A, Dukes B, Dukes C1, or Dukes C2. The results from pooled cancer samples are shown as “All Dukes”.

From FIG. 3A it can be seen that as cancer progresses the amount of NAP1L1 expressed increases. Data were collected from 8 samples in each “normal” or Duke's stage group. The dot-plot analysis shows NAP1L1 concentrations in individual patients, while the horizontal line indicates the mean for each group. Horizontal lines with arrow bars indicate the mean with SEM.

FIG. 3B shows the results of receive operating characteristic (ROC) analysis conducted on the ELISA results depicted in FIG. 3A. It can be seen that when all colorectal cancer samples are taken as a whole (“all stages” result), the area under the ROC curve is 0.8906, and the P value is 0.0007271, clearly indicating that expression is elevated in colorectal cancer samples as compared to controls, and that this elevation is statistically significant.

Taqman quantitative real-time PCR analysis was undertaken using conventional protocols as follows. Briefly, total RNA from human colorectal cancer and mouse tissue samples were used to synthesize first strand cDNA using a Verso™ cDNA Kit (Thermo Scientific) and anchored oligo-dT primers (Thermo Scientific) according to the manufacturer's instructions.

qRT-PCR—Single-stranded cDNA samples were amplified in a polymerase chain reaction using sequence-specific primers (Eurogentec) and probes from the Universal Probe Library (Roche) that were designed using the Universal ProbeLibrary Assay Design Centre, using PCR Master mix (Roche) and a light cycler 480 (Roche).

Human primers and probes used are set out in Appendix C of this application.

Fold change in NAP1L1 expression were corrected with respect to beta-actin expression in the same samples (delta Ct). Levels of expression in tumour samples were compared with those observed in adjacent normal tissue, and these results were subject to statistical analysis. The results, and a summary of the statistical analysis, are shown in FIG. 3C. Briefly it can be seen that mRNA encoding NAP1L1 (a target molecule representative of expression of this gene for the purposes of the present application) is increased approximately two-fold in colorectal cancer samples as compared to controls. There is little overlap between the expression levels in the two sample types, and these differences are statistically significant (P<0.0001).

In view of the above, it will be recognised that methods in accordance with the first aspect of the invention, in which elevated expression of NAP1L1 is used as a biomarker, are highly suited to detection of cancer of the colon, or precancerous conditions of the colon.

### 2 Analysis of Further Biomarkers for Use in Detection of Cancer or a Precancerous Condition

Taqman quantitative real-time PCR analysis was undertaken using the protocols described above to individually assess the levels of target molecules representative of expression of:
RPL6
PHB
CK18
HMGB1
FABP6
NPM
   found in normal and colorectal cancer samples.

All colorectal cancer tissues and adjacent uninvolved colonic mucosa were obtained from surgically removed specimens with informed patient consent. Uninvolved colonic mucosa was generally 5-10 cm away from any malignant tissue. These samples are from a variety of stages Dukes A-C2.

As above, fold change in NAP1L1 expression were corrected with respect to beta-actin expression in the same samples (delta Ct). Levels of expression in tumour samples were compared with those observed in adjacent normal tissue, and these results were subject to statistical analysis.

The results, and a summary of the statistical analysis, are shown in Panels A to F of FIG. 4. In these it can be seen that mRNA (a nucleic acid target molecule) representative of expression of each of the biomarkers tested is increased in colorectal cancer samples as compared to controls. In each case there is little overlap between the expression levels in the two sample types, and these differences are statistically significant.

### 3 PHB and RPL6 as Biomarkers for Cancer, or Precancerous Conditions, of the Colon

The results reported above illustrate the utility of NAP1L1 as a biomarker for the detection of cancer, or precancerous conditions, of the colon. Similar utilities for PHB and RPL6 were confirmed in the following further study conducted using human serum samples from patients with colorectal cancer, or from control individuals.

Sandwich ELISAs for PHB or RPL6 were undertaken using sandwich enzyme immunoassays purchased from Uscn Life Science Inc, and the experimental protocols described above.

The results obtained from sandwich ELISAs for PHB and RPL6 are respectively shown in FIGS. 5 and 6.

For each biomarker, serum concentration (indicative of expression) is assessed in controls (labelled “normal”) and colorectal cancer patients at the following stages of cancer development: Dukes A; Dukes B; Dukes C1; and Dukes C2. Concentrations from the samples of all pooled colorectal cancer patients are also shown, as “All Dukes”.

Panels B and C of each of FIGS. 4 and 5 show the results of ROC analysis. Panels B of FIGS. 4 and 5 respectively illustrate ROC analysis results for PHB or RPL6 in the pooled cancer patient samples.

Panel C of FIG. 4 shows ROC results for PHB expression in patients staged at Dukes B. Area under the ROC curve is 0.7969, Standard Error 0.1202; 95% confidence interval 0.5613 to 1.032; P value 0.04605. The data were generated from eight control samples and 8 patient samples.

Panel C of FIG. 5 shows ROC results for RPL6 expression in patients staged at Dukes C. Area under the ROC curve is 0.8661, Standard Error 0.09864; 95% confidence interval 0.6727 to 1.059; P value 0.01771. These data were generated from eight control samples and seven patient samples.

Further analysis of the contribution of each of the biomarkers NAP1L1, PHB and RPL6 to methods utilising combinations of these biomarkers are discussed in section 12 below.

### 4 Detection of Cancer Via Assay for Target Molecules Present in Blood Samples

The following study demonstrated that expression of the biomarkers used in methods of the present invention may be investigated in blood samples (i.e. substantially whole blood) as well as in serum samples. It will be appreciated that whole blood represents a useful alternative form of patient sample suitable for use in the methods of the invention, and may offer advantages over plasma samples. Many of these arise from the nature of the target molecules contained in the different sample types, since serum samples will generally be used as a source of protein target molecules (such as the expressed biomarker proteins themselves), while blood samples will generally serve as a source of nucleic acid target molecules (such as mRNA indicative of expression in cells within the blood). Relative benefits of protein and nucleic acid target molecules are discussed further elsewhere in the specification.

In the present study gene expression was detected by real time PCR conducted on nucleic acid target molecules extracted from cells present in circulating blood. As detailed below, in one embodiment directly extracted RNA was assessed for gene expression, and in an alternative embodiment RNA extracted from the cells was subject to an amplification technique prior to assaying for elevated levels of the target molecules. Embodiments in which RNA extracted from blood cells was subject to amplification prior to assaying allowed the detection of statistically significant changes in expression of NAP1L1 and PHB in blood samples from mice with the model of colorectal cancer, as opposed to control mice. These results illustrate that such techniques are likely to provide an important tool in the detection or staging of cancer or precancerous conditions.

Briefly, murine blood samples (approximately 0.5 ml in volume) taken from AhCre⁺Apc^{fl/fl} mice and AhCre⁺Apc^{f/+} mice, and RNA was extracted from these using the PAXgene Blood RNA Kit by PreAnalytix/Qiagen (Hombrechtikon).

Where appropriate, RNA target molecules within the samples were amplified and converted to cDNA using the MessageBOOSTER™ Whole Transcriptome cDNA Synthesis Kit for qPCR by epicentre (Madison). Non-amplified samples were converted to cDNA using First Strand cDNA synthesis kit for RT-PCR (AMV) by Roche, (Sussex).

qRT-PCR primers were designed using ProbeFinder and synthesised by Eurogentec (Southampton). qRT-PCR was performed using the LightCycler® 480 System by Roche, (Sussex). CP value data processing was performed using GenEX. Statistical analysis was performed using SigmaPlot 11.1. Box plates were made using SPSS Statistics 20.

The results of these studies are set out in FIGS. 7, 8 and 9, which respectively show results of assays for elevated levels of target molecules representative of expression of NAP1L1, PHB and CK18.

The results of statistical analysis of assays for non-amplified target molecules indicative of NAP1L1 expression (shown in FIG. 7) are as follows:

### Mann-Whitney U Statistic=36.000

T=90.000 n(small)=9 n(big)=9 (P=0.724)

These results indicate that, in the sample size investigated, difference in the median values between the two groups is not great enough to exclude the possibility that the difference is due to random sampling variability; there is not a statistically significant difference (P=0.724)

In contrast the results of statistical analysis of assays for amplified target molecules indicative of NAP1L1 expression (shown in FIG. 6) are as follows:

t=3.586 with 4 degrees of freedom. (P=0.023)
95 percent confidence interval for difference of means: 0.0231 to 0.181

The difference in the mean values of the two groups is greater than would be expected by chance; there is a statistically significant difference between the input groups (P=0.023).

FIG. 8 shows the results for assays conducted on amplified or non-amplified target molecules representative of PHB expression. The results of statistical analysis of these assays were as follows:

### Non-Amplified Samples:

t=1.519 with 16 degrees of freedom. (P=0.148)
95 percent confidence interval for difference of means: −0.0290 to 0.175

The difference in the mean values of the two groups is not great enough to reject the possibility that the difference is due to random sampling variability. There is not a statistically significant difference between the input groups (P=0.148).

### Whereas for Amplified Samples:

t=3.604 with 4 degrees of freedom. (P=0.023)
95 percent confidence interval for difference of means: 0.0291 to 0.224

The difference in the mean values of the two groups is greater than would be expected by chance; there is a statistically significant difference between the input groups (P=0.023).

FIG. 9 shows the results for assays conducted on amplified or non-amplified target molecules representative of CK18 expression. The results of statistical analysis of these assays were as follows:

### Non-Amplified Samples:

### Mann-Whitney U Statistic=0.000

T=126.000 n(small)=9 n(big)=9 (P=<0.001)

The difference in the median values between the two groups is greater than would be expected by chance; there is a statistically significant difference (P=<0.001)

### Whilst for Amplified Samples:

t=7.624 with 4 degrees of freedom. (P=0.002)
95 percent confidence interval for difference of means: 0.254 to 0.545

The difference in the mean values of the two groups is greater than would be expected by chance; there is a statistically significant difference between the input groups (P=0.002).

Though the results above were obtained using PCR and nucleic acid target molecules, the inventors have found that these results are also observed in assays, such as ELISAs, which investigate the level of protein target molecules present in a sample.

15 Detection of Markers in Serum of Colon Cancer Patients, and Comparison with Normal Serum

The inventors investigated expression of a panel of biomarkers including NAP1L1 (specifically CK18, PHB, NCL, NAP1L1, RPL6, FABP6, NPM, and SFRS2) in patient samples from colon cancer patients and in matched controls. The results of this investigation are shown in FIG. 9, which compares control samples (labelled “Normal”) with samples from cancer patients.

The patient samples used were serum samples, which were assayed by enzyme linked immunosorbent assay (ELISA) for the presence of the various proteins referred to. Thus, in these assays the proteins themselves represented target molecules indicative of expression of the genes by which the proteins are encoded.

As can be seen from FIG. 10 each of the biomarkers investigated exhibited increased expression when compared to the level of expression occurring in a control sample. The error bars shown on the bar charts indicate that in the majority of these cases the difference between normal and cancer expression was statistically significant.

Assays were undertaken using commercially available ELISA kits (listed in the accompanying Appendix) according to the manufacturers' instructions.

Further assays investigating expression of the biomarker HMGB1 were undertaken using a commercially available ELISA kit (ST51011—available from IBL International GmbH). Data from these assays are not shown.

6 Detection of Markers in Serum of Colon Cancer Patients, and Comparison with Normal Serum—Analysis with Reference to Dukes' Stage Disease Progression

Patient samples from colon cancer patients at various stages of the disease (assessed using Dukes staging from A to C2) were compared with serum samples from normal patients without cancer. n=8 patients were investigated for each stage of colorectal cancer.

As before, assays were conducted using commercially available ELISA kits to investigate protein target molecules within serum samples.

The results of this study are illustrated in FIG. 11. Here it can be seen that, in pooled results gathered for the eight patients sampled for each stage of cancer, each of the markers was increased in cancer patients as compared to controls. Furthermore, the results indicate that, when taken as a whole, each biomarker was elevated in patients with colorectal cancer at Dukes' stage B. The biomarkers NAP1L1 and RPL6 were also expressed at notably increased levels in samples from patients with colorectal cancer at Dukes' stage A. These findings indicate that the panel of biomarkers identified, as well as specific sub-groups within this broader panel, are useful in staging of cancers, and may be of particular use in the staging of cancers at early stages of disease progression.

Further assays investigating expression of the biomarker HMGB1 were undertaken using a commercially available ELISA kit (ST51011—available from IBL International GmbH). Data from these assays are not shown.

7 Comparison of Expression of Biomarkers with Patient Survival

FIG. 11 illustrates a comparison between expression of the biomarkers PHB, CK18, NCL, NAP1L1, RPL6, FABP6, SFRS2, or NPM and the length of patient survival. Biomarker expression and survival were compared between colorectal cancer patients (designated “CRC”) and corresponding samples taken from patients without cancer (“Normal”).

The analysis shown in FIG. 12 was undertaken using freely available cluster and treeview software (available, for example, from: http://rana.lbl.gov/EisenSoftware.htm). The red colour referred to shows as lighter blocks in the black and white version of this Figure, with increasing lightness indicating a stronger red colour.

The results obtained illustrate that biomarkers CK18 and PHB are associated with poor survival rates, and so bad prognosis for patients. The exception is in patients were SFRS2 is expressed. This biomarker provides a strong indication that a patient will have a good survival rate, and so a good prognosis, and appears to overcome the drawbacks otherwise associated with CK18 and/or PHB expression.

In light of the above, it will be appreciated that, while HMGB1 may be a specified as one of the genes the expression of which is to be investigated in the methods in accordance with a number of the aspects of the invention, similar methods may be undertaken in which investigation of HMGB1 is omitted, as long as CK18 and PHB are both investigated.

### 8 Utility of PHB and CK18 as Biomarkers for the Detection of Cancer Requiring Aggressive Therapeutic Regimens

As set out elsewhere in the specification, certain subsets of colorectal cancer patients have a form of the disease that is associated with shortened survival times. Patients having this form of colorectal cancer thus require more aggressive treatment than other patients.

Elevated expression of one or more of the biomarkers PHB and/or CK18 provides an indication that the cancer in question is likely to require treatment using a more aggressive therapeutic regimen.

FIGS. 13 and 14 illustrate the results of analysis of expression of PHB and CK18 (respectively) plotted against patient survival. As can be seen elevated expression of either PHB (for example to a level above 12 ng/ml) or CK18 (such as to over 6 ng/ml) is associated with a decrease in patient survival. Statistical analysis (in particular the “logrank” results) indicates that there is a statistically significant decrease in the survival of patients with elevated expression of either of these biomarkers, as opposed to the survival of patients lacking such elevated expression.

9 Detection of Markers in Tumour Tissue Samples of Colon Cancer Patients, and Comparison with Adjacent Normal Tissue—mRNA Results

The expression of biomarkers HMGB1, NCL, CK18, DDX5, PHB, RPL6, SFRS2, CBX3, NLM, NAP1L1 and FABP6 was investigated in tumour samples from patients with colorectal cancer, and expression in tumour samples compared with expression in adjacent normal tissue. These tissue samples, containing biological cells, were investigated using a PCR-based assay in which mRNA constituted the target molecules representative of gene expression.

In brief, RNA samples were provided by the Liverpool Tissue Bank. The PCR was performed using a Roche light cycler 480, with Master mix for human qRT: LightCycler® 480 Probes Master—1st strand kit for human qRT: Thermo Scientific Verso™ cDNA Synthesis Kit (AB-1453). All kits and reagents were used according to manufacturers' directions.

Details of the probes used are provided in the accompanying Appendix. The probe numbers refer to the sequence specific probe from the Universal ProbeLibrary Set, Human (probes 1-90) catalogue number 04683633001 from Roche.

The results in FIG. 15 show expression in tumour samples as a fold-increase over the normal tissue control. These illustrate that cancer samples also exhibit characteristic increases in expression of the biomarkers investigated in assays in which RNA is used as a target molecule representative of gene expression.

10 Comparison of Biomarker Expression as Compared by mRNA-Based Assays and Patient Survival

The expression of biomarkers NCL, CK18, DDX5, PHB, RPL6, SFRS2, CBX3, NLM, NAP1L1 and FABP6 was investigated in tumour samples from patients with colorectal cancer, using a PCR-based assay in which mRNA provided the target molecules representative of gene expression.

FIG. 16 shows the results of a comparison of biomarker expression with patient survival. As can be seen, the results of this study illustrate once more the links between the biomarkers and patient survivals, and hence the prognostic utility of these biomarkers. As with FIG. 12, the red colour referred to shows as lighter blocks in the black and white version of FIG. 16, with increasing lightness indicating a stronger red colour.

### 11 Therapeutic Use of HMGB1 Inhibitors

The effectiveness of inhibitors of HMGB1 activity in therapeutic uses, and particularly the prevention of gastrointestinal tract cancer formation was illustrated in an animal model using mice genetically predisposed to the development of symptoms corresponding to the early stages of colorectal cancer.

Briefly, in APC^{fl/fl} mice the major colorectal cancer gene APC is deleted in the epithelial cells of the intestine. As a result, these mice develop a phenotype associated with early stage colorectal cancer. FIG. 17 shows the results of an investigation into gene expression within the intestinal epithelia of these mice, which illustrates that there is a marked increase in HMGB1 expression.

One of the characteristic of the intestines of such mice is an increase in the length of crypts formed. The cells lining the crypt also exhibit increased levels of cell division (mitosis) and apoptosis.

The inventors have found that provision of an inhibitor of HMGB1 activity is able to reduce the cancer-like effects noted in the intestines of APC^{fl/fl} mice. Anti-HMGB1 Polyclonal Antibody Chicken (IgY Fraction) ST326052233 (IBL International GmbH) was injected intraperitoneally into APC^{fl/fl} mice, and the effects on the intestines investigated.

The agent provided inhibits the cytokine activity of HMGB1, and this is distinct to previous suggestions as to therapeutic uses of inhibitors of HMGB1's transcription factor activity.

FIG. 18 shows that treatment of APC^{fl/fl} mice with an anti-HMGB1 antibody, capable of inhibiting the cytokine effect of HMGB1, is able to reduce crypt length, and also to restore levels of cell division and apoptosis to near normal levels found in wild type (WT) mice with or without control antibody. In contrast, in mice treated with control antibody (IgY that does not inhibit the cytokine activity of HMGB1) crypt length, cell division and cell death are all increased, indicating that it is the ability to inhibit cytokine activity of HMGB1 that gives rise to the beneficial effects noted.

In the graph comparing the results of Ki67 and BRDU labelling, Ki67 is shown as the left-hand bar of each pair, while BRDU is shown as the right-hand bar.

### 12 Combined Use of NAP1L1, PHB and RPL6 as a Panel of Biomarkers for Cancer, or Precancerous Conditions, of the Colon

Principal component analysis (PCA) was used to illustrate the effectiveness of panels of biomarkers comprising NAP1L1, PHB and RPL6 in the detection and staging of cancer and/or precancerous conditions. The PCA analysis also allowed investigation of the contribution made by the different biomarkers to the effectiveness of the panel as a whole. The results of these analyses, which were generated by ELISA assays conducted on serum samples are shown in FIGS. 19 to 22. Here the bar charts provided in FIGS. 19 and 20 show the relative contribution of a range of biomarkers (NCL, PHB, CK18, NAP1L1, SFRS2, FABP6, RPL6, and NPM) including NAP1L1 and PHB to the ability to distinguish between control samples, and samples derived from cancer patients. Statistical significance is indicated by bars that do not cross the 0 line, although other bars that fail to reach statistical significance may still be indicative of some contribution by the biomarker in question.

FIG. 19 shows the results of PCA based on expression of a panel of biomarkers in samples (“normal”/“N”) and in samples from a group of patients with colorectal cancer (“C”). For the purposes of the results shown in FIG. 19, no distinction was made between the staging of cancer patients.

It can be seen from the bar charts set out in FIG. 19 that NAP1L1 and RPL6 both make statistically significant contributions to the ability of the tested panel of biomarkers to discriminate between individuals with and without cancer. It will be appreciated that these results undertaken in respect of the pooled group of cancer patients replicate the circumstance that will most frequently be found in practice, where it is desired to detect whether or not an individual has cancer, without any prior staging having taken place.

FIG. 20 shows the results of PCA based on expression of a panel of biomarkers in samples (“normal”/“N”) and in samples from a group of patients with colorectal cancer staged as Duke's C (“C”). It can be seen from the bar charts set out in this FIG. 20 that NAP1L1 and PHB both make statistically significant contributions to the ability of the tested panel of biomarkers to discriminate between individuals with Duke's C colorectal cancer, and those without cancer. Although RPL6 does not make a statistically significant contribution to this discrimination, the change in expression levels observed for this biomarker does show a trend towards the overall contribution.

FIG. 21 shows the results of PCA based on expression of RPL6 and NAP1L1 in normal samples and samples from Duke's stage B colorectal cancer. These data are represented by various means, but perhaps the most readily interpreted is the “box” at the bottom of the Figure. Here it can be seen that expression in the normal (“N”) and Duke's B cancer (“B”) groups is distinct from one another, as illustrated by the gap between the respective groups of data points.

A similar result is shown in FIG. 22, which illustrates the results of PCA based on expression of RPL6 and NAP1L1 in normal samples (“N”) and samples from Duke's stage C colorectal cancer (“C”). Once again, a distinction can be made between expression levels in each group, as shown by the gap between the groups of data points.

TABLE 1
Approximate frequency and 5 year relative survival (%) by Dukes' stage

| Dukes Stage | Approximate Frequency at | |
|---|---|---|
| (modified) | Diagnosis | Approximate 5-year survival |
| A | 11% | 83% |
| B | 35% | 64% |
| C | 26% | 38% |
| D | 29% | 3% |

TABLE 2
11 potential biomarkers with their expression during the validation process
using samples from the AhCre⁺Apc^{fl/fl}mice model.

| | | | | Western | |
|---|---|---|---|---|---|
| | | iTRAQ | | blot | qPCR |
| | | Fold | IHC | Fold | Fold |
| Name | Description | change | Location | change | change |
| HMGB1 | high-mobility group | 1.546 | Yes | 1.87 | 2.58* |
| | (nonhistone | | | | |
| | chromosomal) | | | | |
| | protein 1-like 1 | | | | |
| NCL | Nucleolin | 1.529 | Yes | 5.94 | 5.35* |
| KRT18 | keratin 18 | 1.455 | Yes | 5.41 | 1.95 |
| RPL6 | ribosomal protein | 1.436 | Yes | 2.16 | 1.22 |
| | L6 | | | | |
| DDX5 | DEAD (Asp-Glu- | 1.425 | Yes | 2.66 | 1.63* |
| | Ala-Asp) box | | | | |
| | polypeptide 5 | | | | |
| PHB | Prohibitin | 1.51 | Yes | NS | 3.19* |
| SFRS2 | splicing factor, | 1.162 | Yes | NS | 2.34* |
| | arginine/serine- | | | | |
| | rich 2 | | | | |
| FABP6 | Fatty acid binding | 12.50 | NS | NS | 1.46 |
| | protein-ileal | | | | |
| NAP1L1 | Nucleasome | 1.71 | NS | 2.66 | 5.51** |
| | assembly protein 1- | | | | |
| | like 1 | | | | |
| NPM | Nucleophosmin | 1.45 | Yes | 2.93* | 3.47* |
| CBX3 | Chromobox protein | 1.27 | Yes | 2.16* | 2.49** |
| | homolog 3 | | | | |

(fold change observed in the iTRAQ analysis; apparent increase as detected by IHC in the “Apc floxed zone”; WB—western blot; qRT PCR real-time PCR; (NS = non specific antibody, ND = not done. * = statistically significant (qRT-PCR)) observed for the initial 9 candidate proteins. Six proteins (highlighted in bold) were identified as potential biomarkers requiring further study.

APPENDIX
A Protein nomenclature
It will be appreciated that protein nomenclature is subject to change. Although we believe
that the nomenclature used in the present disclosure is clear in and of itself, the following
information is provided in an attempt to further assist the skilled person in understanding the
invention.
All Aliases & Descriptions and external IDs referred to below are provided from come from
Genecard (http://www.genecards.org/)
A1 HMGB1 is used to refer to “high-mobility group box 1”, which is also known by the
following aliases, descriptions and external identifications
Aliases & Descriptions

| | |
|---|---|
| high-mobility group box 1¹² | Amphoterin² |
| HMG1²³⁵ | OTTHUMP00000018197² |
| DKFZp686A042361¹² | OTTHUMP00000018198² |
| HMG3¹² | Sulfoglucuronyl carbohydrate |
| | binding protein² |
| high-mobility group (nonhistone chromosomal) | high mobility group box 1² |
| protein 1¹² | |
| SBP-1¹² | high mobility group protein B1² |
| High mobility group protein 1²³ | OTTHUMP00000190859² |
| HMG-1²³ | |

| | | | | |
|---|---|---|---|---|
| External | HGNC: | Entrez Gene: | Ensembl: | UniProtKB: |
| Ids: | 4983¹ | 3146² | ENSG00000189403⁷ | P09429³ |

A2 RPL6 is used to refer to “ribosomal protein L6”, which is also known by the following
aliases, descriptions and external identifications
Aliases & Descriptions

| | |
|---|---|
| ribosomal protein L6¹² | 60S ribosomal protein L6² |
| TAXREB107¹² | DNA-binding protein TAXREB107² |
| Tax-responsive enhancer element-binding protein 107²³ | SHUJUN-2² |
| Neoplasm-related protein C140²³ | TaxREB107³ |
| TXREB1²³ | |

| | | | | |
|---|---|---|---|---|
| External | HGNC: | Entrez Gene: | Ensembl: | UniProtKB: |
| Ids: | 10362¹ | 6128² | ENSG00000089009⁷ | Q02878³ |

A3 NCL is used to refer to “nucleolin”, which is also known by the following aliases,
descriptions and external identifications
Aliases & Descriptions
nucleolin¹²
C23¹²
FLJ45706²
FLJ59041²
Protein C23³

| | | | | |
|---|---|---|---|---|
| External | HGNC: | Entrez Gene: | Ensembl: | UniProtKB: |
| Ids: | 7667¹ | 4691² | ENSG00000115053⁷ | P19338³ |

A4 PHB is used to refer to “prohibitin”, which is also known by the following aliases,
descriptions and external identifications
Aliases & Descriptions

| | |
|---|---|
| prohibitin¹² | OTTHUMP00000220171² |
| PHB1¹² | OTTHUMP00000165765² |
| OTTHUMP00000220170² | OTTHUMP00000165766² |
| OTTHUMP00000220103² | |

| | | | | |
|---|---|---|---|---|
| External | HGNC: | Entrez Gene: | Ensembl: | UniProtKB: |
| Ids: | 8912¹ | 5245² | ENSG00000167085⁷ | P35232³ |

A5 NPM is used to refer to “nucleophosmin”, which is also known by the following
aliases, descriptions and external identifications
Aliases & Descriptions

| | |
|---|---|
| nucleophosmin (nucleolar phosphoprotein B23, | numatrin² |
| numatrin)¹² | |
| NPM¹²³ | MGC104254² |
| B23¹² | nucleophosmin² |
| Nucleolar protein NO38²³ | Numatrin³ |
| nucleophosmin/nucleoplasmin family, member 1² | Nucleolar phosphoprotein B23³ |

| | | | | |
|---|---|---|---|---|
| External | HGNC: | Entrez Gene: | Ensembl: | UniProtKB: |
| Ids: | 7910¹ | 4869² | ENSG00000181163⁷ | P06748³ |

A6 CK18 is used to refer to “cytokeratin 18”, which is also known by the following
aliases, descriptions and external identifications
Aliases & Descriptions

| | |
|---|---|
| keratin 18¹² | cytokeratin 18² |
| Cell proliferation-inducing gene 46 protein²³ | keratin, type I cytoskeletal 18² |
| K18²³ | keratin-18² |
| CYK18²³ | cell proliferation-inducing protein 46² |
| CK-18²³ | Cytokeratin-18³ |
| cytokeratin-18² | Keratin-18² |

| | | | | |
|---|---|---|---|---|
| External | HGNC: | Entrez Gene: | Ensembl: | UniProtKB: |
| Ids: | 6430¹ | 3875² | ENSG00000111057⁷ | P05783³ |

A7 NAP1L1 is used to refer to “nucleosome assembly protein 1-like 1”, which is also
known by the following aliases, descriptions and external identifications
Aliases & Descriptions

| | |
|---|---|
| nucleosome assembly protein 1-like 1¹² | hNRP²³ |
| NRP¹²³ | NAP-1-related protein²³ |
| NAP1L¹²⁵ | FLJ16112² |
| NAP1¹² | HSP22-like protein interacting protein² |
| MGC23410¹² | NAP-1 related protein² |
| MGC8688¹² | |

| | | | | |
|---|---|---|---|---|
| External | HGNC: | Entrez Gene: | Ensembl: | UniProtKB: |
| Ids: | 7637¹ | 4673² | ENSG00000187109⁷ | P55209³ |

A8 SFRS2 is used to refer to “serine/arginine-rich splicing factor 2”, which is also known
by the following aliases, descriptions and external identifications
Aliases & Descriptions

| | |
|---|---|
| serine/arginine-rich splicing factor 2¹² | SFRS2A¹² |
| Splicing factor, arginine/serine-rich 2¹²³ | Splicing factor SC35²³ |
| SC-35¹²³ | Splicing component, 35 kDa²³ |
| SFRS2²³⁵ | SR splicing factor 2² |
| PR264¹² | SRp30b² |
| SC35¹² | Protein PR264³ |

| | | | | |
|---|---|---|---|---|
| External | HGNC: | Entrez Gene: | Ensembl: | UniProtKB: |
| Ids: | 10783¹ | 6427² | ENSG00000161547⁷ | Q01130² |

A9 FABP6 is used to refer to “fatty acid binding protein 6”, which is also known by the
following aliases, descriptions and external identifications
Aliases & Descriptions

| | |
|---|---|
| fatty acid binding protein 6, ileal¹² | Intestinal bile acid-binding protein²³ |
| ILLBP¹²³⁵ | Intestinal 15 kDa protein²³ |
| ILBP¹²³ | Ileal lipid-binding protein²³ |
| I-15P¹²³ | Fatty acid-binding protein 6²³ |
| I-BABP¹²³ | GT²³ |
| I-BALB¹² | illeal lipid-binding protein² |
| ILBP3¹² | gastrotropin² |
| I-BAP¹² | ileal bile acid binding protein² |

| | | | | |
|---|---|---|---|---|
| External | HGNC: | Entrez Gene: | Ensembl: | UniProtKB: |
| Ids: | 3561¹ | 2172² | ENSG00000170231⁷ | P51161² |

A10 DDX5 is used to refer to “DEAD box polypeptide 5”, which is also known by the
following aliases, descriptions and external identifications
Aliases & Descriptions

| | |
|---|---|
| DEAD (Asp-Glu-Ala-Asp) box polypeptide 5¹² | DKFZp434E109² |
| HLR1²³⁵ | probable ATP-dependent RNA helicase |
| | DDX5² |
| G17P1²³⁵ | DEAD box-5² |
| DEAD/H (Asp-Glu-Ala-Asp/His) box polypeptide 5 | DKFZp686J01190² |
| (RNA helicase, 68 kD)¹² | |
| p68¹² | ATP-dependent RNA helicase DDX5² |
| RNA helicase p68²³ | EC 3.6.4.13² |
| DEAD box protein 5²³ | HELR³ |
| HUMP68² | |

| | | | | |
|---|---|---|---|---|
| External | HGNC: | Entrez Gene: | Ensembl: | UniProtKB: |
| Ids: | 2746¹ | 1655² | ENSG00000108654⁷ | P17844³ |

A11 CBX3 is used to refer to “chromobox homologue 3”, which is also known by the
following aliases, descriptions and external identifications
Aliases & Descriptions

| | |
|---|---|
| chromobox homolog 3¹² | chromobox protein homolog 3² |
| HP1Hs-gamma¹² | heterochromatin protein HP1 gamma² |
| Modifier 2 protein²³ | chromobox homolog 3 (HP1 gamma homolog, |
| | Drosophila)² |
| Heterochromatin protein 1 homolog | heterochromatin-like protein 1² |
| gamma²³ | |
| HECH²³ | OTTHUMP00000202463² |
| chromobox homolog 3 (Drosophila HP1 | HP1 gamma homolog² |
| gamma)¹ | |
| HP1-GAMMA² | HP1 gamma³ |

| | | | | |
|---|---|---|---|---|
| External | HGNC: | Entrez Gene: | Ensembl: | UniProtKB: |
| Ids: | 1553¹ | 11335² | ENSG00000122565⁷ | Q13185³ |

B ELISA kits used in generating Experimental Results
These kits were used in accordance with the manufacturer's instructions.
B1 Human ELISA kits
ELISA Kit for Human Keratin 18 (KRT18) E91231Hu
ELISA Kit for Human Nucleosome Assembly Protein 1 Like Protein 1 (NAP1L1) E97571Hu
ELISA Kit for Human Splicing Factor, Arginine/Serine Rich 2 (SFRS2) E95102Hu
ELISA Kit for Human Nucleophosmin (NPM) E92664Hu
ELISA Kit for Human Nucleolin (NCL) E92242Hu
ELISA Kit for Human Prohibitin (PHB) E90442Hu
ELISA Kit for Human Ribosomal Protein L6 (RPL6) E95046Hu
ELISA Kit for Fatty Acid Binding Protein 6, Ileal (FABP6) E90344Hu
B2 Mouse ELISA kits
ELISA Kit for Mouse Chromobox Homolog 3(CBX3) E2363 Mu ELISA Kit for Mouse
Prohibitin(PHB) E0442 Mu ELISA Kit for Mouse Nucleophosmin(NPM)E2664 Mu ELISA Kit
for Mouse Fatty Acid Binding Protein 6, Ileal(FABP6)E0344 Mu
ELISA Kit for Mouse Nucleolin(NCL) E2242 Mu
The above kits were produced by:
Uscn Life Science Inc.Wuhan
108, Zhuanyang Avenue, Economic & Technological Development Zone, Wuhan, 430056
P.R.China
B3 HMGB1 ELISA for use on mouse and human
ST51011 from IBL International GmbH
C Primers used for analysis of gene expression via mRNA target molecules

| Gene | Probe # | Forward | Reverse |
|---|---|---|---|
| NCL | #80 | GTG-GAT-GTC-AGA-ATT-GGT-ATG-ACT | CAA-ACC-AGT-GAG-TTC-CAA-CG |
| NPM | #69 | CGG-ATG-ACT-GAC-CAA-GAG-G | TTA-CAG-AAA-TGA-AAT-AAG-ACG-GAA-AA |
| DDX5 | #69 | GCC-ATG-TCG-GGT-TAT-TCG | GGT-TTC-CAA-ACT-TCT-TTC-CAG-A |
| PHB | #31 | ACC-TTC-GGG-AAG-GAG-TTC-AC | CGC-CTT-TTT-CTG-TTG-CTC-A |
| NAP1L1 | #35 | TGA-TCA-AGA-TTT-GGA-TGA-TGT-TG | AAC-AGT-TAG-CTG-ACG-TGC-TTT-G |
| CBX3 | #86 | TTT-GCC-AGA-GGT-CTT-GAT-CC | TGC-CTC-ATC-TGA-ATC-TTT-CCA-T |
| FABP6 | #3 | AGG-AGA-CCT-GCA-GAG-AAT-GAA | CAC-TCT-CCA-TCT-CGA-ACT-TGC |
| SFRS2 | #65 | TTG-GCC-AGT-ATT-GCA-GAT-TTT | CAA-TAC-ACG-CCC-ACA-GAA-AC |
| CK18 | #78 | TGA-TGA-CAC-CAA-TAT-CAC-ACG-A | GGC-TTG-TAG-GCC-TTT-TAC-TTC-C |
| RPL6 | #73 | ATT-ACG-GAG-CAG-CGC-AAG | CCA-TTC-GTC-AGA-GCA-AAC-AC |
| HMBG1 | #76 | GAG-TGA-GGA-GGC-TGC-GTC-T | TGC-CCA-TGT-TTA-GTT-ATT-TTT-CC |

1. A method of detecting a cancer of the colon, or a precancerous condition of the colon, the method comprising assaying a patient sample for an elevated level of target molecules representative of expression of nucleosome assembly protein 1-like 1 (NAP1L1), wherein an elevated level of the target molecules representative of the expression of NAP1L1 is indicative of a cancer of the colon, or a precancerous condition of the colon.

2. A method according to claim 1, further comprising assaying the patient sample for an elevated level of a further target molecule representative of expression of a gene selected from the group consisting of: RPL6; and PHB, wherein elevated levels of the target molecules representative of the expression of NAP1L1 and the further target molecules are indicative of a cancer of the colon, or a precancerous condition of the colon.

3. A method according to claim 2, comprising assaying the patient sample for further target molecules representative of expression of RPL6 and further target molecules representative of expression of PHB.

4. A method of detecting a cancer or a pre-cancerous condition, the method comprising:
assaying a patient sample for an elevated level of target molecules representative of expression of at least two genes encoding proteins selected from the group consisting of: HMGB1; PHB; RPL6; NAP1L1 and CK18;
wherein the elevated level of the target molecules is indicative of a cancer or pre-cancerous condition in the patient.

5. A method according to claim 4, wherein the cancer to be detected is an early stage cancer.

6. A method for assessing effectiveness of a therapy or putative therapy, the method comprising:
determining a level of expression of NAP1L1 in a sample representative of gene or protein expression in a sample of a cancer;
providing the therapy or putative therapy to a sample of a cancer;
determining a level of expression of NAP1L1 in a sample representative of gene expression in a sample of the cancer to which the therapy or putative therapy has been provided.

7. A method according to claim 6, further comprising determining levels of expression of one or more further genes or proteins selected from the group consisting of: PHB and/or RPL6.

8. A method of detecting cancer or a pre-cancerous condition, the method comprising:
assaying a patient sample for an elevated level of target molecules representative of expression of at least two genes encoding proteins selected from the group consisting of: HMGB1; PHB; and CK18;
wherein an elevated level of the target molecules in the patient sample is indicative of a cancer or pre-cancerous condition in the patient.

9. A method according to claim 8, further comprising assaying a patient sample for an elevated level of a target molecule representative of expression of the gene encoding FABP6.

10. A method according to claim 8, wherein an elevated level of the target molecule representative of gene expression is assessed by comparing the amount of the target molecule present in the patient sample under investigation with a reference value indicative of the amount of the target molecule in a control sample.

11. A method of detecting cancer or a pre-cancerous condition, the method comprising:
assaying a patient sample for an elevated level of target molecules representative of expression of the group of genes consisting of:
HMGB1, RPL6, NCL, PHB, NPM, CK18, NAP1L1, SFRS2, FABP6, DDX5, CBX3
wherein the presence of the target molecules representative of expression of these genes is indicative of a cancer or pre-cancerous condition in the patient.

12. A method of staging a cancer, the method comprising:
assaying a sample from a patient with cancer for the presence of target molecules representative of expression of at least two genes encoding protein selected from a group comprising:
HMGB1, RPL6, NCL, PHB, CK18, NAP1L1, FABP6, DDX5, and CBX3
wherein an elevated level of the target molecule representative of expression of the genes indicates the stage of the patient's cancer.

13. A method of staging a cancer, the method comprising:
assaying a sample from a patient with cancer for the presence of target molecules representative of expression of a gene encoding a protein selected from a group comprising:
HMGB1, RPL6, NCL, PHB, CK18, NAP1L1, FABP6, DDX5, CBX3
and using the information regarding the expression of these genes to stage the patient's cancer.

14. A method according to claim 13, further including include assaying the patient sample for target molecules representative of expression of NPM and/or SFRS2.

15. A method according to claim 12, wherein, an increase in expression across the sum of the genes assessed is indicative that the patient's cancer is at a stage equivalent to stage B of the Dukes' classification system for colorectal cancer.

16. A method according to claim 12, wherein an increase in expression of genes selected from a comprising HMGB, NCL, PHB and CK18 is indicative that the patient's cancer is at a stage equivalent to Dukes C.

17. A method according to claim 12, wherein an increase in expression of genes selected from a subgroup comprising NAP1L1; RPL6 and HMGB1 may be indicative that the patient's cancer is at a stage equivalent to stage A of the Dukes' classification system.

18. A method of staging a cancer, the method comprising:
assaying a sample from a patient with cancer for the presence of target molecules representative of expression of a gene encoding protein selected from a group comprising:
HMGB1, RPL6, and NAP1L1,
and using the information regarding the expression of these genes to stage the patient's cancer.

19. A method of determining a prognosis for survival of a cancer patient, the method comprising:
assaying a sample from a patient with cancer for the presence of target molecules representative of expression of SFRS2 and/or NPM;
wherein an elevated level of the target molecules in the sample is indicative of a good prognosis.

20. A method of determining a prognosis for survival of a cancer patient, the method comprising:
assaying a sample from a patient with cancer for the presence of target molecules representative of expression of:
SFRS2 and/or NPM; and
HMGB1 and/or PHB and/or CK18
wherein a decreased level of the target molecules representative of expression of SFRS2 and/or NPM, and an increased level of the target molecules representative of expression of PHB and/or CK18 is indicative of a poor prognosis.

21. A method of selecting a cancer treatment regimen, the method comprising:
assaying a sample from a patient with cancer for the presence of target molecules representative of expression of:
SFRS2 and/or NPM; and
HMGB1 and/or PHB and/or CK18
wherein a decreased level of the target molecules representative of expression of SFRS2 and/or NPM, and an increased level of the target molecules representative of expression of PHB and/or CK18 indicates that the patient would benefit from an aggressive cancer treatment regimen.

22. A method according to claim 21, wherein the aggressive treatment regimen includes surgery to remove much or all of the tissue in which cancer is present.

23. A method according to claim 4, wherein the cancer, or pre-cancerous condition, is Wnt-driven.

24. A method according to claim 4, wherein the cancer, or pre-cancerous condition, is Wnt-driven cancer of the digestive tract, Wnt-driven breast cancer, Wnt-driven lung cancer, Wnt-driven liver cancer, Wnt-driven ovarian cancer, Wnt-driven neurological cancers, or Wnt-driven skin cancer.

25. A method according to claim 1, wherein the patient sample is a serum sample.

26. A method according to claim 2, wherein the patient sample is a blood sample.

27. A method according to claim 1, wherein the target molecule is the expressed protein encoded by the gene.

28. A method according claim 1, wherein the assay for the target molecule is selected from a group comprising: enzyme linked immunosorbant assays (ELISA), including variants such as sandwich ELISAs; radioimmuno assays (RIA); immunocytochemistry labelling; immunohistochemistry labelling of a tissue sample; fluorescence activated cell sorting (FACS); chemiluminescence; and multiplex assays such as Luminex or proteomic MRM.

29. A method according to claim 1, wherein the target molecule is RNA encoding the protein.

30. A method according to claim 29, wherein the assay for the target molecule comprising reverse transcription PCR (rt PCR) of the RNA.

31. An inhibitor of HMGB1 activity for use as a medicine for the prevention of cancer of the digestive tract.

32. An inhibitor of HMGB1 activity for use according to claim 31, for use as a medicament for provision to patients with a pre-cancerous condition.

33. The inhibitor of HMGB1 activity according to claim 31, which is an inhibitor of the cytokine activity of HMGB1.

34. The inhibitor of HMGB1 activity according to claim 31, which is an extracellular inhibitor of HMGB1 activity.

35. The inhibitor of HMGB1 activity according to claim 31, comprising a neutralising antibody.


SEQUENCE LISTING (22 sequences)

SEQ ID NO: 1 - DNA, 24 bp, Artificial
  Forward primer for NCL
gtggatgtcagaattggtatgact

SEQ ID NO: 2 - DNA, 20 bp, Artificial
  Reverse primer for NCL
caaaccagtgagttccaacg

SEQ ID NO: 3 - DNA, 19 bp, Artificial
  Forward primer for NPM
cggatgactgaccaagagg

SEQ ID NO: 4 - DNA, 26 bp, Artificial
  Reverse primer for NPM
ttacagaaatgaaataagacggaaaa

SEQ ID NO: 5 - DNA, 18 bp, Artificial
  Forward primer for DDX5
gccatgtcgggttattcg

SEQ ID NO: 6 - DNA, 22 bp, Artificial
  Reverse primer for DDX5
ggtttccaaacttctttccaga

SEQ ID NO: 7 - DNA, 20 bp, Artificial
  Forward primer for PHB
accttcgggaaggagttcac

SEQ ID NO: 8 - DNA, 19 bp, Artificial
  Reverse primer for PHB
cgcctttttctgttgctca

SEQ ID NO: 9 - DNA, 23 bp, Artificial
  Forward primer for NAP1L1
tgatcaagatttggatgatgttg

SEQ ID NO: 10 - DNA, 22 bp, Artificial
  Reverse primer for NAP1L1
aacagttagctgacgtgctttg

SEQ ID NO: 11 - DNA, 20 bp, Artificial
  Forward primer for CBX3
tttgccagaggtcttgatcc

SEQ ID NO: 12 - DNA, 22 bp, Artificial
  Reverse primer for CBX3
tgcctcatctgaatctttccat

SEQ ID NO: 13 - DNA, 21 bp, Artificial
  Forward primer for FABP6
aggagacctgcagagaatgaa

SEQ ID NO: 14 - DNA, 21 bp, Artificial
  Reverse primer for FABP6
cactctccatctcgaacttgc

SEQ ID NO: 15 - DNA, 21 bp, Artificial
  Forward primer for SFRS2
ttggccagtattgcagatttt

SEQ ID NO: 16 - DNA, 20 bp, Artificial
  Reverse primer for SFRS2
caatacacgcccacagaaac

SEQ ID NO: 17 - DNA, 22 bp, Artificial
  Forward primer for CK18
tgatgacaccaatatcacacga

SEQ ID NO: 18 - DNA, 22 bp, Artificial
  Reverse primer for CK18
ggcttgtaggccttttacttcc

SEQ ID NO: 19 - DNA, 18 bp, Artificial
  Forward primer for RPL6
attacggagcagcgcaag

SEQ ID NO: 20 - DNA, 20 bp, Artificial
  Reverse primer for RPL6
ccattcgtcagagcaaacac

SEQ ID NO: 21 - DNA, 19 bp, Artificial
  Forward primer for HMGB1
gagtgaggaggctgcgtct

SEQ ID NO: 22 - DNA, 23 bp, Artificial
  Reverse primer for HMGB1
tgcccatgtttagttatttttcc